(19) United States
(12) Patent Application Publication
(10) Pub. No.: US 2024/0170163 A1
(43) Pub. Date: May 23, 2024

(54) SYSTEMS AND METHODS FOR DETECTING NEW DRUG PROPERTIES IN TARGET-BASED DRUG-DRUG SIMILARITY NETWORKS

(71) Applicant: University of Southern California, Los Angeles, CA (US)

(72) Inventors: Paul Bogdan, Los Angeles, CA (US); Lucretia Udrescu, Los Angeles, CA (US); Mihai Udrescu, Los Angeles, CA (US)

(21) Appl. No.: 18/280,919

(22) PCT Filed: Jan. 10, 2022
(86) PCT No.: PCT/US2022/011864

§ 371 Date: Sep. 7, 2023

Related U.S. Application Data
(60) Provisional application No. 63/159,932, filed on Mar. 11, 2021.

(51) Int. Cl. G16H 70/40

(52) U.S. Cl. CPC G16H 70/40

(57) ABSTRACT

A method includes generating topological clusters and network communities, relating each cluster and each community to a pharmacological property or pharmacological action, identifying, within each topological cluster or modularity class community, a subset of drugs that are not compliant with the cluster or community label, validating indicated repositionings, and analyzing molecular docking parameters for previously unaccounted repositionings.

### CROSS-REFERENCE TO RELATED APPLICATIONS

The present disclosure claims the benefit of and priority to U.S. Provisional Application No. 63/159,932, filed Mar. 11, 2021, the disclosure of which is incorporated herein by reference in its entirety.

### GOVERNMENT FUNDING STATEMENT

This invention was made with government support under N66001-17-1-4044 awarded by Defense Advanced Research Projects Agency (DARPA) and 1453860 awarded by the National Science Foundation (NSF). The government has certain rights in the invention.

### BACKGROUND

The present disclosure relates generally to the field of computational drug screening. More particularly, the present disclosure relates to systems and methods for detecting or uncovering new drug properties in target-based drug-drug similarity networks.

Accurately predicting drug properties can be difficult. For example, due to the complexity of biological environments, knowledge of chemical structures cannot fully explain the nature of interactions between drugs and biological targets.

### SUMMARY

At least one aspect relates to a method including generating topological clusters and network communities, relating each cluster and each community to a pharmacological property or pharmacological action, identifying, within each topological cluster or modularity class community, a subset of drugs that are not compliant with the cluster or community label, validating indicated repositionings, and analyzing molecular docking parameters for previously unaccounted repositionings.

At least one aspect relates to a method including generating, by one or more processors, a drug-drug similarity network; determining, by the one or more processors, at least one of a cluster or a community using the drug-drug similarity network; and determining, by the one or more processors, a repositioning of at least one drug associated with the drug-drug similarity network.

At least one aspect relates to a method. The method includes generating, by one or more processors using a plurality of characteristics of relationships between a plurality of drugs and a plurality of biological components, a network including a plurality of nodes and a plurality of edges, each edge of the plurality of edges connecting a respective first node of the plurality of nodes and a respective second node of the plurality of nodes, the respective first node corresponding to a respective first drug of the plurality of drugs, the respective second node corresponding to a respective second drug of the plurality of drugs, each edge generated based on (1) at least one first characteristic of the plurality of characteristics corresponding to the respective first drug and at least one first biological component of the plurality of targets and (2) at least one second characteristic of the plurality of characteristics corresponding to the respective second drug and the at least one first biological component; identifying, by the one or more processors, a subset including at least a first identified node, a second identified node, and a third identified node of the plurality of nodes; identifying, by the one or more processors, a particular characteristic of the subset, the drug of the third node not assigned the particular characteristic; and storing, by the one or more processors, an association between the particular characteristic and at least one of the third identified node and the drug of the third identified node.

At least one aspect relates to a system including one or more processors. The one or more processors are configured to generate, using a plurality of characteristics of relationships between a plurality of drugs and a plurality of biological components, a network comprising a plurality of nodes and a plurality of edges, each edge of the plurality of edges connecting a respective first node of the plurality of nodes and a respective second node of the plurality of nodes, the respective first node corresponding to a respective first drug of the plurality of drugs, the respective second node corresponding to a respective second drug of the plurality of drugs, each edge generated based on (1) at least one first characteristic of the plurality of characteristics corresponding to the respective first drug and at least one first biological component of the plurality of targets and (2) at least one second characteristic of the plurality of characteristics corresponding to the respective second drug and the at least one first biological component; identify a subset comprising at least a first identified node, a second identified node, and a third identified node of the plurality of nodes; identify a particular characteristic of the subset, the drug of the third node not assigned the particular characteristic; and store an association between the particular characteristic and at least one of the third identified node and the drug of the third identified node.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification.

### BRIEF DESCRIPTION OF THE DRAWINGS

The accompanying drawings are not intended to be drawn to scale. Like reference numbers and designations in the various drawings indicate like elements. For purposes of clarity, not every component can be labeled in every drawing. In the drawings:

FIG. 1 depicts a chart of new drug applications (NDAs) and new molecular entities (NMEs) during 1940-2017.

FIGS. 2A-2C depict examples of using drug-target interaction information to generate a drug-drug similarity network.

FIG. 3 depicts an example of a DDSN generated using methods described herein, where the node colors identify distinct modularity clusters.

FIG. 4 depicts an example of a zoomed detail view of a DDSN of Community 1 (C_1, Antineoplastic drugs—Mitotic inhibitors & DNA-damaging anticancer drugs), in which the red arrows indicate the reconstructed drug repositionings: Colchicine (antigout drug), Podofilox (topical antiviral), and Enoxacin, Ciprofloxacin, Moxifloxacin, Gatifloxacin (fluoroquinolone antibiotics).

FIG. 5 depicts an example of a zoomed detail view of a DDSN that highlights Mimosine's presence (an experimental antineoplastic which inhibits DNA replication) in C₆, which can indicate that Mimosine has effects in hormone-dependent cancers.

FIG. 6 depicts an example of a zoomed detail view of a DDSN that highlights the presence of Fenofibrate and Amiloride in C3, which can indicate that the highlighted drugs also have anti-inflammatory effects.

FIG. 7 depicts an example of a zoomed detail view of a DDSN that highlights the presence of Isoflurane and Methoxyflurane in C_25; this indicates that the highlighted drugs may also have antifungal effects.

FIGS. 8A-8D depict examples of a power-law distributions of centrality parameters in the drug-drug similarity network (DDSN).

FIG. 9 depicts an example of a DDSN based on drug-target interactions.

FIG. 10 depicts an example of a summary of interactions resulted from the molecular docking analysis of drug-target pairs.

FIG. 11 depicts an example of a summary of interactions resulted from the molecular docking analysis of drug-target pairs.

FIG. 12 depicts an example of a system to generate DDSNs.

FIG. 13 depicts an example of a method of generating DDSNs.

### DETAILED DESCRIPTION

Systems and methods as described herein can use unsupervised machine learning and information regarding drug-target, drug-gene, and/or drug-side effect interactions to infer drug properties. A weighted drug-drug similarity network (DDSN) can be built based on drug-drug similarity relationships defined based on various such interactions. Using an energy-model network layout or other clustering processes described herein, drug communities can be generated that are associated with specific, dominant drug properties, indicating potential repurposing, including repositioning, for drugs based on cluster membership. For an example of such a DDSN, DrugBank confirms the properties of 59.52% of the drugs in these communities, and 26.98% correspond with drug repositioning indicators determined using the DDSN. The remaining 13.49% of the drugs can be classified as candidate drug repositioning indicators based on such drugs not matching dominant pharmacologic properties. Using the DDSN can reduce computational requirements to more efficiently and accurately detect at least one of repositionings of drugs or properties of drugs. The DDSN can be used to filter or screen candidate drugs for further evaluation by in silico, in vitro, and/or in vivo testing, improving the drug development pipeline.

A prioritizer can be applied, based on betweenness/degree node centrality, to reduce computational resources for testing the drug repositioning indicators. For example, using betweenness/degree as a measure of drug repurposing potential, Azelaic acid and Meprobamate were identified as a possible antineoplastic and antifungal, respectively. A test procedure based on molecular docking can be used to analyze Azelaic acid and Meprobamate's repurposing.

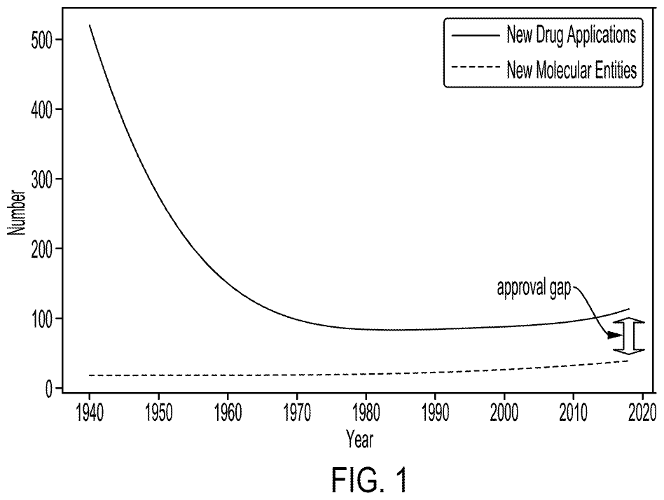

Conventional drug design has become expensive and cumbersome, as it requires large amounts of resources and faces serious challenges. Although the number of new FDA drug applications (NDAs) has significantly increased during the last decade, such as due to accumulation of multi-omics data and the appearance of increasingly complex bioinformatics tools, the number of approved drugs has only marginally grown (see FIG. 1).

Drug repositioning (or drug repurposing) can be used to find new pharmaceutical functions for already used drugs. For example, medical and pharmaceutical experience demonstrate multiple indications for many drugs, including examples based on drug repositioning. For example, out of the 90 newly approved drugs in 2016 (a 10% decrease from 2015), 25% are repositionings in formulations, combinations, and indications. Drug repositioning can reduce the incurred research and development (R&D) time and costs and medication risks.

Computational methods can be useful for drug repositioning based on factors such as the spread of omics analytical approaches that have generated significant volumes of useful multi-omics data (genomics, proteomics, metabolomics, and others); increased access and volume of data on drug-drug interactions and drug side-effects; and developments in physics, computer science, and computer engineering to enable efficient methods and technologies for data exploration and mining, such as complex network analysis, machine learning, or deep learning.

Complex network analysis can be a useful tool for predicting unaccounted drug-target interactions. Network-based computational drug repurposing approaches can use data on confirmed drug-target interactions to predict new such interactions, thus leading to new repositioning indicators. Some approaches build drug-drug similarity networks, where the similarity is defined based on transcriptional responses. These repositioning approaches can analyze the network parameters and the node centrality distributions in either drug-drug or drug-target networks, using statistical analysis and machine learning (e.g., graph convolutional networks) [26-29] to link certain drugs to new pharmacological properties. However, some statistics can be misleading when used to predict extreme centrality values, such as degree and betweenness (which particularly indicate nodes/drugs with a high potential for repositioning). While some network-related approaches introduce useful repositioning pipelines, they are mostly based on multi-partite and multilayered unweighted networks, which can be challenging to process and interpret.

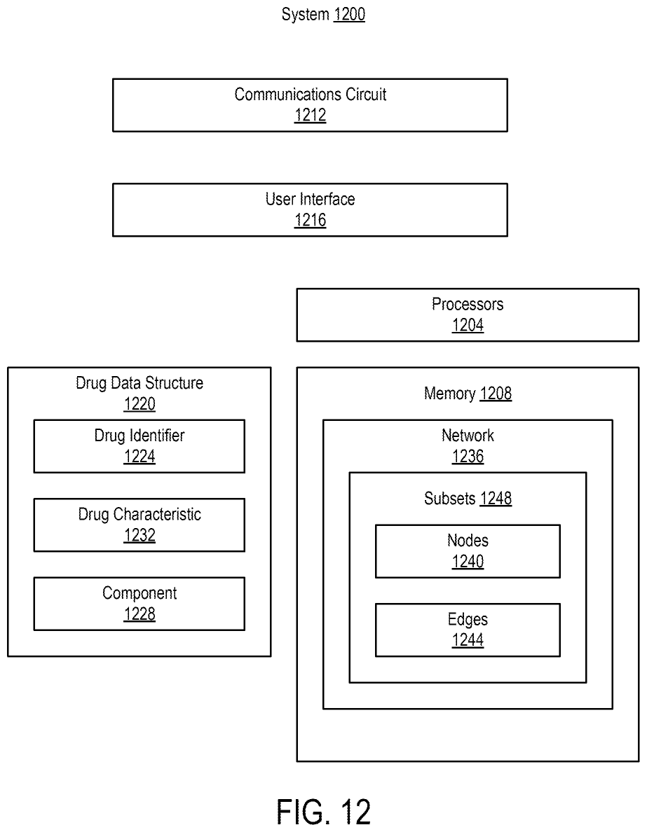

Systems and methods in accordance with the present disclosure can implement a network-based, computational approach to drug repositioning. For example, a weighted drug-drug network can be generated, such as a network where the nodes are drugs, and the weighted links represent relationships between drugs. The network can be based on information from the accurate DrugBank. In the DDSN, a link can be placed between two drugs if their interaction with at least one target is of the same type (either agonistic or antagonistic). The link weight can represent the number of biological targets that interact in the same way with the two drugs. Various processes described herein can be at least partially implemented using a system such as system 1200 described with reference to FIG. 12.

For example, a method can include generating topological clusters and network communities (e.g., using the Force Atlas 2 layout and modularity classes); relating each cluster and each community to a pharmacological property or pharmacological action (e.g., label communities and clusters according to the dominant property or pharmacological action), using expert analysis; identifying and selecting (e.g., by betweenness divided by degree, b/d) within each topological cluster/modularity class community, the top drugs not compliant with the cluster/community label (e.g., using the b/d centrality to find the centrality's distribution, which can be more stable in the DDSN than network analysis that uses centralities to rank nodes; validating the indicated (e.g., hinted) repositionings (e.g., by searching DrugBank); and analyzing molecular docking parameters for previously unaccounted repositionings.

### Drug-Drug Similarity Network

The DDSN can be implemented as a weighted graph G=(V, E), where V is the vertex (or node) set, and E is the edge (or link) set; the vertices (nodes) represent drugs and the edges (links) represent drug-drug similarity relationships based on drug-target interactions. G can have |V| vertices vᵢ∈V and |E| edges e_{j,k}∈E, with i,j,k∈{1,2, . . . |V|} and j≠k. Each edge e_{j,k} is characterized by a weight w(e_{j,k})≠0 (in an unweighted network, w(e_{j,k})=1, ∀e_{j,k}∈E). The DDSN can be implemented using data representing various interactions, such as drug-target, drug-gene, drug-side effect, or various combinations thereof. The interactions can include various interactions representing how drugs interact with biological components, including but not limited to agonist and antagonist interactions.

The weight can represent a degree of target action similarity between drugs vⱼ and vₖ, and it is equal with the number of common biological targets for vⱼ and vₖ, as determined based on the drug-target relationships (e.g., agonist or antagonist relationships between particular drugs and particular targets or other biological components). Consequently, w(e_{j,k})∈ [symbol] *, ∀e_{j,k}∈Ew(ej,k).

If e_{j,k}=0, then there is no target similarity between vⱼ and vₖ, therefore no edge between these nodes. A data structure representing the network can indicate that there is no edge between respective nodes by assigning a value of zero to e_{j,k}, or by not including the edge in E. A common biological target is a target tₖ∈T (T is the set of targets) on which drugs vⱼ and vₖ act in the same way, such as either both agonistically or both antagonistically.

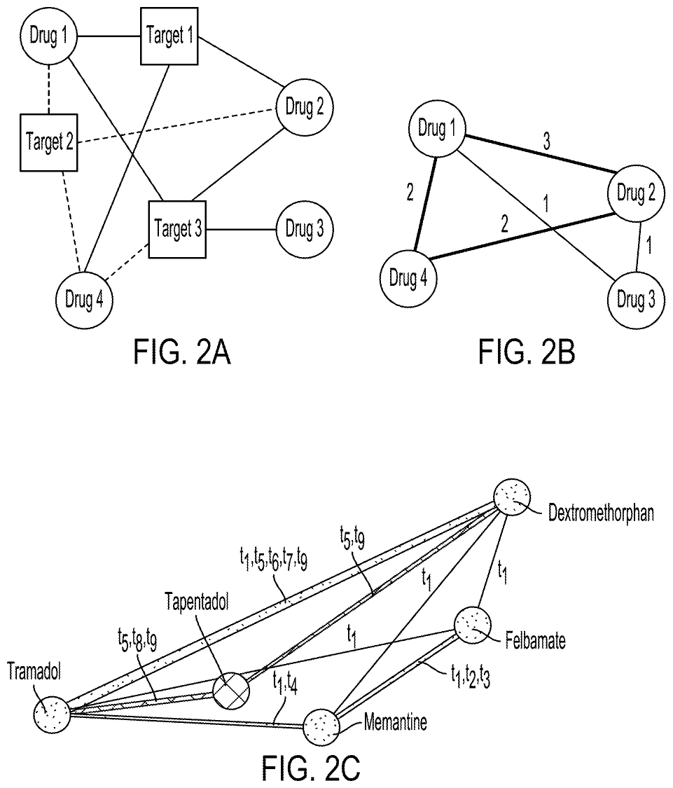

FIG. 2 illustrates an example of generating of the DDSN with information on drug-target interactions. Panel (a) depicts drug-target interactions between four drugs (i.e., round nodes labeled 1 to 4) and three targets (i.e., square nodes labeled 1 to 3). The dashed red links represent agonist drug-target interactions, and the solid blue links represent antagonist drug-target interactions. Panel (b) depicts the DDSN corresponding to the interactions in (a). For instance, a link of weight 3 connects the nodes 1 and 2 because Drug 1 and Drug 2 interact in the same way for the three targets, i.e., agonist on Target 2 and antagonist on Targets 1 and 3. Furthermore, a link with weight 2 connects Drug 2 and Drug 4 because they both interact agonistically on Target 2 and antagonistically on Target 1, but they do not interact in the same way with Target 3. Panel (c) depicts a DDSN sub-network example, according to drug-target interactions from DrugBank 4.2, containing drugs Dextromethorphan, Felbamate, Tapentadol, Tramadol, and Memantine. The link thickness is depicted according to the weight and the list of common targets is specified for each link. The weight equals the number of targets in the list, where t_1=Glutamate receptor ionotropic NMDA 3A, t_2=Glutamate receptor ionotropic NMDA 2A, t_3=Glutamate receptor ionotropic NMDA 2B, t_4=Alpha-7 nicotinic cholinergic receptor subunit, t_5=Mu-type opioid receptor, t_6=Kappa-type opioid receptor, t_7=Delta-type opioid receptor, t_8=Sodium-dependent noradrenaline transporter, and t_9=Sodium-dependent serotonin transporter. FIG. 2 depicts an example of a DDSN in which the biological component is a target; DDSNs can be generated as described herein for various biological components or interactions associated with biological components, including but not limited to genes and side effects

For the DDSN graph G, the drug-target interaction information from Drug Bank 4.2 was used. The analysis was based on the largest connected component of the DDSN, consisting of |V|=1008 drugs/nodes and |E|=17963 links resulted from the analysis of the drug-target interactions with |T|=516 targets. The analysis depicted with respect to FIG. 2 used the Drug Bank version 4.2, such as to allow a different version (e.g., the latest Drug Bank 5.1.4) for testing the accuracy of the drug property prediction.

### Network Analysis

Network analysis can be used to uncover new drug properties from the drug-target data. Network clustering (e.g., network community detection) can be used to associate drugs with previously unaccounted drug properties and network centralities to prioritize the uncovered drug repurposing hints. For example, as described further herein, subsets of nodes in the network, such as clusters of nodes, can be used to predict properties for drugs in the subsets that may not be previously known.

### Network Clustering

The network clustering can classify each node vᵢ∈V in one of the disjoint sets of nodes (cluster Cᵢ⊂V, with i=1̅‥̅m̅, C₁∪C₂ . . . ∪Cₘ=V). Modularity can be used to define the node membership to one of the clusters. Modularity can correspond to an amount of edges in a particular cluster relative to an amount than would be expected if the edges were assigned randomly. The modularity of a clustering Cₘ={C₁, C₂, . . . Cₘ} can be defined as

$${m}_{} = {\sum }_{{C}_{i} ∈ {C}_{m}} ( \frac{\vert {E}_{{C}_{i}} \vert}{\vert E \vert} - \frac{\vert \frac{1}{2} {d}_{{C}_{i}}^{2} \vert}{\vert \frac{1}{2} {d}^{2} \vert} ) \qquad ( 1 )$$

where |E| is the total number of edges in G, |E_{C}_{ᵢ}| is the total number of edges between nodes in cluster Cᵢ, d is the total degree of nodes in G, and d_{C}_{ᵢ} is the total degree of nodes in cluster Cᵢ. Thus,

$$\frac{\vert {E}_{{C}_{i}} \vert}{\vert E \vert}$$

can represent the edge density of cluster Cᵢ relative to the entire network G density, whereas

$$\frac{\frac{1}{2} {d}_{{C}_{i}}^{2}}{\frac{1}{2} {d}^{2}}$$

is the Cᵢ's expected relative density.

The clustering can be performed in various manners. For example, clustering can

be performed using the software package Gephi [38], by maximizing the modularity from Equation (1) with the method introduced and analyzed in references [39,40]. The approach is to divide a graph into two communities, to achieve a target value of modularity, such as maximum modularity. The binary method can then be applied recursively on each resulted community, thus dividing them further; the entire process comes to an end when the overall modularity cannot be further increased. To describe the division algorithm, the graph modularity can be determined as

$$M = \frac{1}{4 k} {\sum }_{ij} ( {A}_{ij} - \frac{{d}_{i} {d}_{j}}{2 k} ) ( {s}_{i} {s}_{j} + 1 ) .$$

In Equation (2), Aᵢⱼ is the graph's adjacency matrix, dᵢ and dⱼ are respectively the degrees of vertices/nodes vᵢ and vⱼ, and k is the total number of edges in the network (k=

$$\vert E \vert = \frac{1}{2} {\sum }_{i} {d}_{i}$$

for an unweighted network). Furthermore, sᵢ=1 if vᵢ is classified in community 1 and sᵢ=−1 if vᵢ is classified in community 2. As such:

$$\frac{1}{2} ( {s}_{i} {s}_{j} + 1 ) = \{ \begin{matrix}1 & if {v}_{i} and {v}_{j} are in the same community \\ 0 & otherwise\end{matrix} \qquad ( 3 )$$

Because the network is weighted, each edge has a weight w(e_{i,j})=w_{i,j}∈ [symbol] *, and Equation (1) can be rewritten as

$${M}_{m} = {\sum }_{{C}_{i} ∈ {C}_{m}} ( \frac{{w}_{{E}_{{C}_{i}}}}{{w}_{E}} - \frac{\frac{1}{2} {w}_{{C}_{i}}^{2}}{\frac{1}{2} {w}_{V}^{2}} ) \qquad ( 4 )$$

In Equation (4), W_{E} is the total edge weight of edges E in G,

$${w}_{{E}_{{C}_{i}}}$$

is the total edge weight of edges in cluster Cᵢ, w_{V} is the total edge weight of all vertices V in G, and w_{C}_{ᵢ} is the total edge weight of vertices in cluster Cᵢ.

In some implementations, the modularity can be used to generate clusters by assigning a cluster to each node, and moving nodes (e.g., reassigning nodes) to different clusters (e.g., adjacent or neighboring clusters) responsive to determining that that the move causes an increase in modularity (e.g., a positive modularity gain, e.g. as shown Eqn. 15 below, corresponding to an overall increase in modularity from the removal of the moved node from a first cluster and the addition of the node to a second cluster). A resolution parameter λ is used to determine whether to move the node from the first cluster to the second cluster; for example, the change in modularity can be compared with λ, and the node can be moved from the first cluster to the second cluster responsive to determining that the change in modularity is greater than (or greater than or equal to) λ, such that a lower value of λ can result in a higher number of resulting clusters. The resolution parameter λ can be set to a predetermined value (e.g., 1), or evaluated using various values of λ to select a particular value of λ to use for generating repositioning hints.

The resolution parameter λ can be used to comparatively evaluate cluster formation and resulting drug repositioning hints, such as by performing a process that includes one or more of the following operations:
1) for λ in range (a to b), with step size c (e.g., for λ greater than or equal to 0.1 and less than or equal to 5, with step size 0.1; various ranges of λ and step sizes can be used):
2) generate a plurality of clusters from the DDSN using λ
3) for each cluster:
4) identify a particular characteristic, e.g. a dominant property, such as a level 1 ATC code assigned to at least a threshold number of nodes of the cluster, e.g. the majority of nodes of the cluster, such as by generating a histogram of level 1 ATC codes for the cluster
5) for each node in the cluster:
6) compare the level 1 ATC codes for the drug of the node with the particular characteristic of the cluster
7) if the particular characteristic (e.g. level 1 ATC code) of the cluster is not included in the level 1 ATC codes of the drug, indicate that the drug is a candidate for repositioning (this can be validated by identifying the level 1 ATC codes from a first database, e.g. a first version of DrugBank, and comparing with the level 1 ATC codes from a second database, e.g. a second version of DrugBank, to determine whether the particular characteristic is included as a level 1 ATC code in the second database)
8) assign the candidate drugs to a list of repositioning candidates
9) evaluate the lists of repositioning candidates for each value of λ to identify λₘₐₓ for the value of λ corresponding to a largest number of repositioning candidates

In an example such process for a DDSN generated from drug-gene interaction data from DrugBank 5.0.9 and compared with data from DrugBank 5.1.8 to validate repositioning candidates, λₘₐₓ was determined to be 2.0. Using various such processes, clusters can be generated from the DDSN based on modularity, and used to identify candidate drugs for repurposing and/or repositioning in a computationally efficient manner.

In some implementations, the network can be arranged into a two-dimensional (2D) space, such as to facilitate energy-based approaches for arranging the nodes and/or arranging clusters of nodes, such as to define communities of nodes to facilitate identifying candidates for repurposing or repositioning. A network layout algorithm places each vertex vᵢ in a 2D space [symbol] × [symbol] = [symbol] ². Therefore, each node vᵢ∈V has its 2D coordinates γᵢ=(xᵢ, yᵢ)∈ [symbol] ², and each edge e_{i,j}∈E has a Euclidian distance δ_{i,j}=|γᵢ−γⱼ|.

In an energy-model, force-directed layout, there can be a force of attraction between any two adjacent nodes vᵢ and vⱼ, and a repulsion force between any two non-adjacent nodes. The expression of these forces is |γᵢ−γⱼ|^{f}\vec{γᵢγ_{J}}, where f=a for attraction and f=r for repulsion. The attraction force between adjacent nodes (vᵢ and vⱼ such that ∃e_{i,j}∈E) decreases, whereas the repulsion force between non-adjacent nodes (vᵢ, vⱼ such that ∃!e_{i,j}∈E) increases with the Euclidian distance. Therefore, a≥0 and r≤0.

The energy-model force-directed layout Force Atlas 2 can be used to assign node positions in the 2D (i.e., [symbol] ²) space, based on interactions between attraction and repulsion forces, such that we attain minimal energy in the network layout,

$$ε = min \{ {\sum }_{( {v}_{i} , {v}_{j} ) , i \neq j} ( \frac{{\vert {γ}_{i} - {γ}_{j} \vert}^{a}}{a + 1} - \frac{{\vert {γ}_{i} - {γ}_{j} \vert}^{r}}{r + 1} ) \} \qquad ( 5 )$$

The energy-based layouts generate topological communities (e.g., clusters) (which can be used to identify candidate drugs for repurposing or repositioning) because specific regions in the network have larger than average link densities. The energy-based topological communities can be equivalent to the network clusters based on modularity classes, when a>−1 and r>−1. Furthermore, given that the DDSN is a weighted network, Equation (5) can be rewritten accordingly, to maintain equivalency with Equation (4),

$$ε = min \{ {\sum }_{( {v}_{i} , {v}_{j} ) , i \neq j} ( {w}_{i , j} \frac{{\vert {γ}_{i} - {γ}_{j} \vert}^{a}}{a + 1} - {w}_{i} {w}_{j} \frac{{\vert {γ}_{i} - {γ}_{j} \vert}^{r}}{r + 1} ) \} \qquad ( 6 )$$

where wᵢ and wⱼ represent the total weight of edges incident to nodes vᵢ and vⱼ (i.e., the weighted degree of vertices vᵢ and vⱼ), respectively, while w_{i,j} is the weight of edge e_{i,j}.

### Network Centralities

Node centralities can be complex network parameters that characterize the vertex/node's importance in a graph. The weighted degree, degree, betweenness, and betweenness/degree node centralities can be evaluated to find that betweenness/degree is appropriate for the prioritizing of drug repositioning hint tests. For example, betweenness/degree centrality can be a crucial driver of complex network dynamics. The degree of the node can correspond to a number of edges connected with the node, and can be weighted by the weights of the edges to the node.

The weighted degree of a node vᵢ is the sum of the weights characterizing the links/edges incident to vᵢ,

d(vᵢ)=Σ_{j∈{x|e}_{_{i,x}}_{∈E,v}_{ₓ}_{,v}_{ᵢ}_{∈V}}w(e_{i,j}). (7)

The degree of a node vᵢ can be computed with Equation (7), assuming that w(e_{i,j})=1, ∀e_{i,j}∈E.

To compute the node betweenness, the shortest paths between all node pairs vⱼ, vₖ can be found in graph G, namely σ_{j,k}. As such, the betweenness of node vᵢ can be the number of minimal paths in graph G that cross node vᵢ, divided by the total number of minimal paths in G,

$$b ( {v}_{i} ) = {\sum }_{( j , k ) ∈ \{ ( x . y ) \vert {v}_{x} \neq {v}_{y} \neq {v}_{i} ; {v}_{x} , {v}_{y} , {v}_{i} ∈ V \}} \frac{{σ}_{j , k} ( {v}_{i} )}{{σ}_{G}} , \qquad ( 8 )$$

where the total number of shortest paths in G is the combinations of 2 vertices from V,

$${σ}_{G} = ( \begin{matrix}\vert V \vert \\ 2\end{matrix} ) . \qquad ( 9 )$$

The betweenness/degree of node vᵢ is the ratio

$$\frac{b}{d ( {v}_{i} )} = \frac{b ( {v}_{i} )}{d ( {v}_{i} )} , \qquad ( 10 )$$

where Equation (7) computes d(vᵢ) in the unweighted version (i.e., considering w(e_{i,j})=1, ∀e_{i,j}∈E).

### Molecular Docking for Repurposing Testing

The effectiveness of the network-based drug repurposing prediction method is emphasized by the fact that DrugBank 4.2 confirms the properties predicted for 59.52% of the drugs, and 26.98% are drug repositioning indicators (e.g., hints) determined using the DDSN approach (confirmed by the later DrugBank 5.1.4 and recent scientific literature). The remaining 13.49% of the drugs can be classified as candidate drug repositioning indicators based on such drugs not matching dominant pharmacologic properties, and which may be candidates for further testing, including the molecular docking simulations presented herein.

### Testing Procedure

To verify the predicted properties of the drug repurposing indicators, molecular docking can be performed for at least one of the drug repurposing indication, a reference drug (e.g., drugs having the predicted property), and one or more drugs with little probability of having the predicted property. For example, a testing procedure can include one or more of the following features:
1. Defining the drug sets to enter the docking process, including drugs that are candidates for having the pharmacological property ϕ( [symbol] ₕ^{ϕ}), drugs with property ϕ (reference drugs [symbol] ᵣ^{ϕ}), and drugs with little probability of having property ϕ( [symbol] ₙ^{ϕ}), such as to identify the similarity (in terms of relevant target activity) between the reference drugs [symbol] ᵣ^{ϕ} and the tested drugs [symbol] ₜ^{ϕ}= [symbol] ₕ^{ϕ}∪ [symbol] ₙ^{ϕ}.
   (a) [symbol] ₕ^{ϕ} includes the drugs that can be candidates for being repurposed for property/properties ϕ.
   (b) [symbol] ᵣ^{ϕ} includes two subsets, reference drugs in the DDSN's community Cₓ( [symbol] ₓ^{ϕ}) and reference drugs not in Cₓ( [symbol] _{x̅}^{ϕ}), with [symbol] ᵣ^{ϕ}= [symbol] ₓ^{ϕ}∪ [symbol] _{x̅}^{ϕ}.
(c) [symbol] ₙ^{ϕ} includes drugs expected to have other pharmacological properties, with little probability of having property ϕ.
2. Establishing the target sets. For pharmacological property ϕ, the targets from DrugBank can be taken into consideration that interact with the drugs in the hinted (e.g., candidate) drug dₕ^{ϕ} community Cₓ having property ϕ( [symbol] ₓ^{ϕ}), and the targets from DrugBank that interact with the drugs with property ϕ not included in DDSN's Cₓ( [symbol] _{x̅}^{ϕ}).
3. For the set of tested drugs [symbol] ₜ^{ϕ}, using molecular docking to check the interactions between all possible drug-target pairs, defined as the Cartesian product of sets [symbol] ₜ^{ϕ} and [symbol] ^{ϕ} (with [symbol] ^{ϕ}= [symbol] ₓ^{ϕ}∪ [symbol] _{x̅}^{ϕ}),

[symbol]

ₜ^{ϕ}× [symbol] ^{ϕ}={(dᵢ, tⱼ):dᵢ∈ [symbol] ₜ^{ϕ}, tⱼ∈ [symbol] ^{ϕ}, ∀i,j∈ [symbol] *, i≤| [symbol] ₜ^{ϕ}|, j≤| [symbol] ^{ϕ}|}. (11)
4. For the set of reference drugs, applying molecular docking on separately designed drug-target pairs for reference drugs in Cₓ( [symbol] ₓ^{ϕ}), and reference drugs not in Cₓ( [symbol] _{x̅}^{ϕ}) respectively, for example such that any drug-target pair is well-documented in the literature,

{(dᵢ, tⱼ): dᵢ∈ [symbol] ₓ^{ϕ}, tⱼ∈ [symbol] ₓ^{ϕ}, ∀i,j∈ [symbol] *, i≤| [symbol] ₓ^{ϕ}|, j≤| [symbol] ₓ^{ϕ}|=1} (12)

and

{(dᵢ, tⱼ): dᵢ∈ [symbol] _{x̅}^{ϕ}, tⱼ∈ [symbol] _{x̅}^{ϕ}, ∀i,j∈ [symbol] *, i≤| [symbol] _{x̅}^{ϕ}|, j≤| [symbol] _{x̅}^{ϕ}|=1}. (13)

In Equations (12) and (13), Boolean function 1 is defined as

$$1 ( i , j ) = \{ \begin{matrix}1 & \begin{matrix}if the interaction between drug \\ {d}_{i} and target {t}_{j} is listed in DrugBank\end{matrix} \\ 0 & otherwise\end{matrix} . \qquad ( 14 )$$

### Ligands and Targets Preparation

All ligands' three-dimensional coordinates can be generated using the Gaussian program suite with the DFT/B3LYP/6-311G optimization procedure.

The X-ray crystal structure of the targets can be retrieved as target.pdb files from the protein databases Protein Data Bank and optimized using the ModRefiner software. As examples, the targets and their corresponding codes are Lanosterol 14-alpha demethylase (4LXJ, resolution 1.9 Å), Intermediate conductance calcium-activated potassium channel protein 4 (6D42, resolution 1.75 Å), Lanosterol synthase (1W6K, resolution 2.1 Å), Squalene monooxygenase (6C6N, resolution 2.3 Å), Ergosterol (2AIB, resolution 1.1 Å), Sodium/potassium-transporting ATPase subunit alpha (2ZXE, resolution 2.4 Å), Tubulin (4U3J, resolution 2.81 Å), Progesterone receptor (1A28, resolution 1.8 Å), Androgen receptor (5JJM, resolution 2.15 Å), Estrogen receptor beta (3OLL, resolution 1.5 Å), Estrogen receptor alpha (1A52, resolution 2.8 Å), Steroid 17-alpha-hydroxylase/17,20 lyase (4NKV, resolution 2.646 Å), and Mineralocorticoid receptor (2OAX, resolution 2.29 Å). The preparation of targets can include adding all polar hydrogens, removing the water, and computing the Gasteiger charge.

### Docking Protocol

Molecular docking analysis can be performed using Autodock 4.2.6 with the molecular viewer and graphical support AutoDockTools.

In the docking protocol, for the protein targets, the grid box can be created using Autogrid 4 with 120 Å×120 Å×120 Å in x, y, and z directions, and 1 Å spacing from the target molecule's center. For steroidal target Ergosterol, the grid box is 30 Å×30 Å 30 Å in x, y, and z directions, with 0.375 Å spacing from the target molecule's center.

For the docking process, the Lamarckian genetic algorithm (Genetic Algorithm combined with a local search) can be used, with a population size of 150, a maximum of 2.5×10⁶ energy evaluations, a gene mutation rate of 0.02, and 50 runs. Default settings can be used for the other docking parameters and performed all the calculations in vacuum conditions. AutoDock results can be outputted in the PyMOL (The PyMOL Molecular Graphics System, Version 2.0 Schrodinger, LLC, New York, NY, USA) and the Discovery Studio (Biovia) molecular visualization system (BIOVIA, Dassault Systèmes, BIOVIA Workbook, Release 2017; BIOVIA Pipeline Pilot, Release 2017, San Diego: Dassault Systèmes, 2019, San Diego, CA, USA).

The performance of Autodock 4.2.6 can be evaluated by redocking and then expressing the results as root-mean-square deviation (RMSD) in Å. Calculations can be performed in one or more iterations (e.g., in duplicate) and the results expressed as averages. The redocking involves the overlapping of the ligands for calculating the RMSD with the Discovery Studio software. A comparative RMSD analysis can be run between Autodock 4.2.6 and AutoDock Vina to assess the docking method's repeatability and reproducibility.

### DDSN Analysis

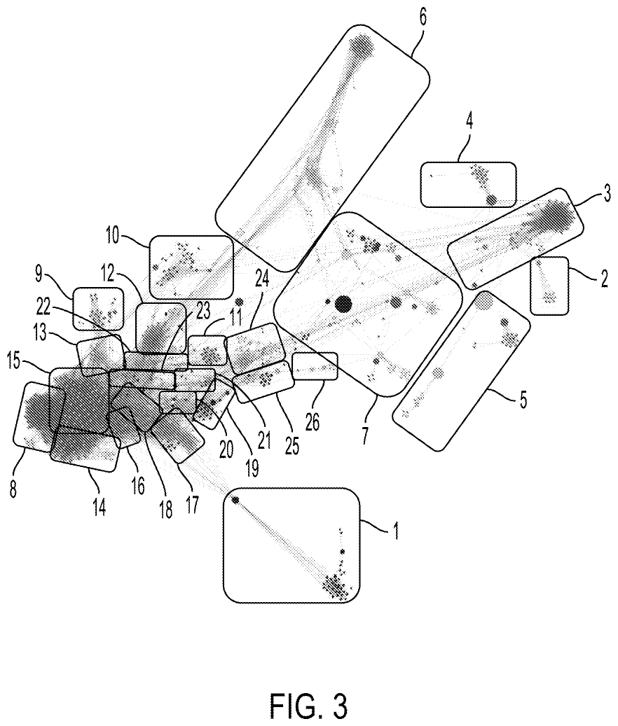

FIG. 3 illustrates the resulted DDSN, built according to our method, where the node colors identify the distinct modularity clusters. For example, for the depicted DDSN, where nodes represent drugs and links represent drug—drug similarity relationships based on drug—target interaction behavior. The layout is Force Atlas 2, and the distinct node colors identify the modularity classes that define the drug clusters. The 26 topological clusters are identified with rounded rectangles and functional descriptions are provided for each.

To mine the DDSN topological complexity, the drug clusters (or communities) can be identified using at least one of the modularity and the force-directed, energy-based layout Force Atlas 2 algorithms. The two clustering techniques are compatible; the energy-based force-directed layout clustering can provide more information about the relationship between clusters and act as an efficient classifier. In the case of DDSN, the clusters can correspond to drug communities Cₓ, x∈ [symbol] *, such that V=═ᵢ₌₁^{m}Cₓ.

Using the constructed DDSN from Drug Bank 4.2 and expert analysis, each cluster can be labeled according to its dominant property (i.e., the property that better describes the majority of drugs in the cluster), which may represent a specific mechanism of pharmacologic action, a specifically targeted disease, or a targeted organ.

When using network clustering, if a drug does not comply with the community/cluster label, then this indicates a possible repurposing. The clusters can be labeled using the drug properties listed by DrugBank or reported in the literature, such that the dominant property or properties (i.e., properties found in more than 50% of the drugs in the community) give the name of the community, as indicated in Tables 1 and 2.

The clusters can be labeled based on receiving labels (e.g., expert labels). The clusters can be labeled using labeling codes in DrugBank or other databases, such as Anatomic Therapeutic Chemical Classification System (ATC) codes, such as Level 1 ATC codes. In some implementations, one or more drugs in a cluster may have multiple labels, such as multiple ATC codes. A histogram can be generated in each cluster based on the labels for the cluster, such as to identify a label having a highest count to assign to the cluster. In some implementations, Level 1 ATC codes are used to reduce computational complexity; various combinations of one or more ATC codes can be used.

According to Tables 1 and 2 (column Literature [%]), the DDSN computational approach recovers/reconstructs a significant number of drug repurposings reported in the literature, namely 26.98% of the 1008 drugs in DDSN (the last line in Table 2, summarizing the confirmation results).

TABLE 1
Confirmation of drug community properties and drug repurposing hints. Each
table line corresponds to a topological community Cₓ (with x = 1̅ .̅ .̅ .̅ 1̅5̅),
by specifying the dominant property (or properties) resulted from the pharmacological
analysis (column Properties), the number of nodes/drugs in community Cₓ
(column Nodes [#]), the percentage of drugs with the properties confirmed
by DrugBank (column DrugBank [%]), the percentage of drugs with the predicted
properties confirmed by the literature (column Literature [%]), the percentage
of drugs with not yet confirmed predicted properties (column Not confirmed [%]),
and the drugs we propose for repositioning, representing predictions not confirmed
yet but with non-zero betweenness/degree in the DDSN (b/d > 0, in column Hints).

| | | | | | Not | |
|---|---|---|---|---|---|---|
| | | Nodes | DrugBank | Literature | Confirmed | |
| Cₓ Properties | | [#] | [%] | [%] | [%] | Hints |
| 1 | Antineoplastic | 37 | 40.54 | 37.84 | 21.62 | Besifloxacin |
| | (mitotic inhibitors | | | | | Pefloxacin |
| | and DNA-damaging) | | | | | Norfloxacin |
| | | | | | | Ofloxacin |
| 2 | Antihypertensive (sartans) | 10 | 100 | 0 | 0 | — |
| 3 | Anti-inflammatory | 84 | 65.48 | 28.57 | 5.95 | Glipizide |
| 4 | Antibacterial tetracyclines | 20 | 95.00 | 0 | 5.00 | Plerixafor |
| | and Aminoglycosides | | | | | |
| 5 | Platelet aggregation | 29 | 10.34 | 82.76 | 6.90 | — |
| | inhibitor | | | | | |
| 6 | Interfering with hormone- | 93 | 26.88 | 65.59 | 7.53 | Azelaic ac. |
| | dependent cancers | | | | | |
| 7 | Anticancer (molecularly | 92 | 23.91 | 50.00 | 26.09 | Suramin |
| | targeted) | | | | | Acetohydroxamic |
| | | | | | | ac. Glyburide |
| | | | | | | Gliquidone |
| | | | | | | Tolbutamide |
| 8 | Anti-allergic | 51 | 86.27 | 11.76 | 1.96 | Butriptyline |
| 9 | Acting on muscles | 25 | 72.00 | 16.00 | 12.00 | — |
| 10 | Vasodilator | 37 | 48.65 | 24.32 | 27.03 | Tofisopam |
| | | | | | | Mefloquine |
| | | | | | | Oxtriphylline |
| | | | | | | Enprofylline |
| | | | | | | Roflumilast |
| | | | | | | Aminophylline |
| 11 | Antiepileptic, | 19 | 84.21 | 10.53 | 5.26 | Barbituric |
| | hypnotic, and | | | | | ac. deriv. |
| | sedative | | | | | |
| 12 | Analgesic and used | 46 | 89.13 | 8.70 | 2.17 | — |
| | in opiate withdrawal | | | | | |
| | & side-effects | | | | | |
| 13 | Antihypertensive, | 26 | 92.31 | 3.85 | 3.85 | — |
| | anti-arrhythmic, | | | | | |
| | anti-angina (mostly | | | | | |
| | beta-blockers) | | | | | |
| 14 | Anticholinergic | 53 | 100 | 0 | 0 | — |
| 15 | Interfering with | 97 | 42.27 | 42.27 | 15.46 | Doxazosin |
| | Parasympathetic | | | | | Terazosin |
| | Nervous System | | | | | Prazosin |
| | | | | | | Paliperidone |
| | | | | | | Aripiprazole |
| | | | | | | Fenoldopam |
| | | | | | | Dapiprazole |
| | | | | | | Alfuzosin |
| | | | | | | Tamsulosin |
| | | | | | | Silodosin |
| | | | | | | Amisulpiride |
| | | | | | | Carphenazine |
| | | | | | | Acetophenazine |

TABLE 2
Confirmation of drug community properties and drug repurposing hints.
Each table line corresponds to a topological community Cₓ (with
x = 1̅6̅ .̅ .̅ .̅ 2̅6̅), as well as the last line for the entire
DDSN, by specifying the dominant property (or properties) resulted from the
pharmacological expert analysis (column Properties), the number of nodes/drugs
in community Cₓ (column Nodes [#]), the percentage of
drugs with the properties confirmed by DrugBank (column DrugBank [%]),
the percentage of drugs with the predicted properties confirmed by the literature
(column Literature [%]), the percentage of drugs with not yet confirmed
predicted properties (column Not confirmed [%]), and the drugs we propose
for repositioning, representing predictions not confirmed yet but with non-zero
betweenness/degree in the DDSN (b/d > 0, in column Hints).

| | | | | | Not | |
|---|---|---|---|---|---|---|
| | | Nodes | DrugBank | Literature | Confirmed | |
| Cₓ Properties | | [#] | [%] | [%] | [%] | Hints |
| 16 | Antidepressant and | 26 | 92.31 | 7.69 | 0 | — |
| | Central Nervous | | | | | |
| | System stimulant | | | | | |
| 17 | Sympathetic Nervous | 61 | 85.25 | 8.20 | 6.56 | — |
| | System acting | | | | | |
| 18 | Antimigraine and | 26 | 42.31 | 26.92 | 30.77 | Captodiame |
| | antiemetic | | | | | Ropinirole |
| | | | | | | MDMA |
| | | | | | | Dofetilide |
| | | | | | | Rotigotine |
| | | | | | | L-DOPA |
| 19 | Antiarrhythmic and | 24 | 66.67 | 12.50 | 20.83 | Acarbose |
| | anticonvulsant | | | | | |
| 20 | Antidepressant and | 21 | 57.14 | 14.29 | 28.57 | Quinidine |
| | anti-Parkinson | | | | | Propafenone |
| | | | | | | Cinchocaine |
| | | | | | | MMDA |
| | | | | | | Aprindine |
| 21 | Interfering with | 12 | 41.67 | 25.00 | 33.33 | Miconazole |
| | epilepsy and | | | | | Quinidine |
| | blood pressure | | | | | barbiturate |
| 22 | Antihypertensive | 20 | 80.00 | 15.00 | 5.00 | — |
| | and anticonvulsant | | | | | |
| 23 | Anesthetic, | 19 | 73.68 | 5.26 | 21.05 | Sympathetic |
| | analgesic, and | | | | | Nervous |
| | muscle relaxant | | | | | System acting |
| 24 | Interfering with | 51 | 50.98 | 13.73 | 35.29 | Progabide |
| | K, Na, Ca | | | | | Bethanidine |
| | homeostasis | | | | | Ellagic ac. |
| | | | | | | Vigabatrin |
| | | | | | | Ethinamate |
| 25 | Antifungal | 22 | 59.09 | 9.09 | 31.82 | Meprobamate |
| | | | | | | Enflurane |
| | | | | | | Sevoflurane |
| | | | | | | Desflurane |
| 26 | Hypnotic and | 7 | 100 | 0 | 0 | — |
| | sedative | | | | | |

| | | | | | |
|---|---|---|---|---|---|
| All- | 1008 | 59.52 | 26.98 | 13.49 | — |

### Illustrative Examples of Reconstructed Drug Repositionings

### Reconstructed Repurposings as Antineoplastic Agents

The topological community 1 (i.e., C₁) includes antineoplastic drugs, mostly mitotic inhibitors (e.g., Etoposide, Teniposide, Vincristine, Vinorelbine) and DNA-damaging anticancer drugs (e.g., Doxorubicin, Valrubicin, Mitoxantrone). This community also includes fluoroquinolone antibiotics (targeting the alpha subunits of two types of bacterial topoisomerase II enzymes, namely DNA gyrase and DNA topoisomerase 4) and a few other drugs. DrugBank does not confirm some drugs' anticancer effects within topological C₁, yet the literature confirms them as such. For example, Colchicine, which is currently used based on its anti-inflammatory effects as an antigout drug, exhibits anticancer effects; Podofilox, a drug for topical treatment of external genital warts, is a potent cytotoxic agent in chronic lymphocytic leukemia (CLL); for some fluoroquinolone drugs, the literature reports anticancer effects (e.g., Enoxacin, Ciprofloxacin, Moxifloxacin, Gatifloxacin).

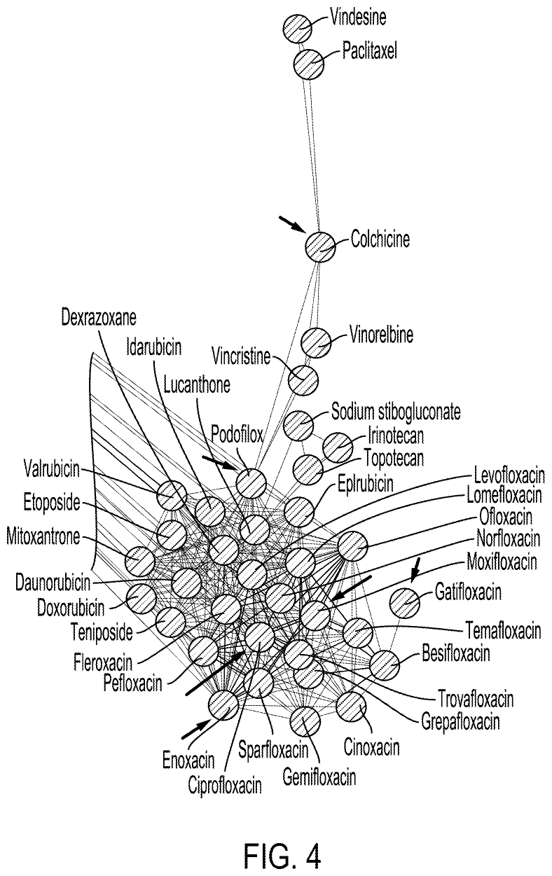

FIG. 4 depicts a zoomed detail from the DDSN, by highlighting the presence of Colchicine, Podofilox, Enoxacin, Ciprofloxacin, Moxifloxacin, Gatifloxacin in C₁; such topological placement suggests their antineoplastic effect.

The topological community C₆ consists of anticancer drugs that target hormone-dependent organs (i.e., ovary, endometrium, vagina, cervix, and prostate). In this community, Progesterone has the highest value of betweenness/degree ratio, and the DrugBank database does not indicate its anticancer property. Although there are extensive epidemiological studies that link the long-term Progesterone use in oral contraceptives to breast cancer risk, this link is strengthened or weakened by various parameters, such as body weight, age, duration of use, parity, age at first birth, breastfeeding, and age at menarche. However, J. C. Leo et al. determined the whole genomic effect of Progesterone in PR-transfected MDA-MB-231 cells and found that Progesterone suppressed the expression of genes involved in cell proliferation and metastasis, concluding that Progesterone can exert a strong anticancer effect in hormone-independent breast cancer following Progesterone receptor (PR) reactivation.

Quinacrine is an antiprotozoal drug that exhibits an anticancer effect in breast cancer because it produces apoptosis by blocking cells in S-phase, induces DNA damage, and inhibits topoisomerase activity; indeed, the clinical trial test of Quinacrine may be recommended for the treatment of patients with androgen-independent prostate cancer. The antineoplastic drug Mimosine attenuates cell proliferation of prostate carcinoma cells in vitro.

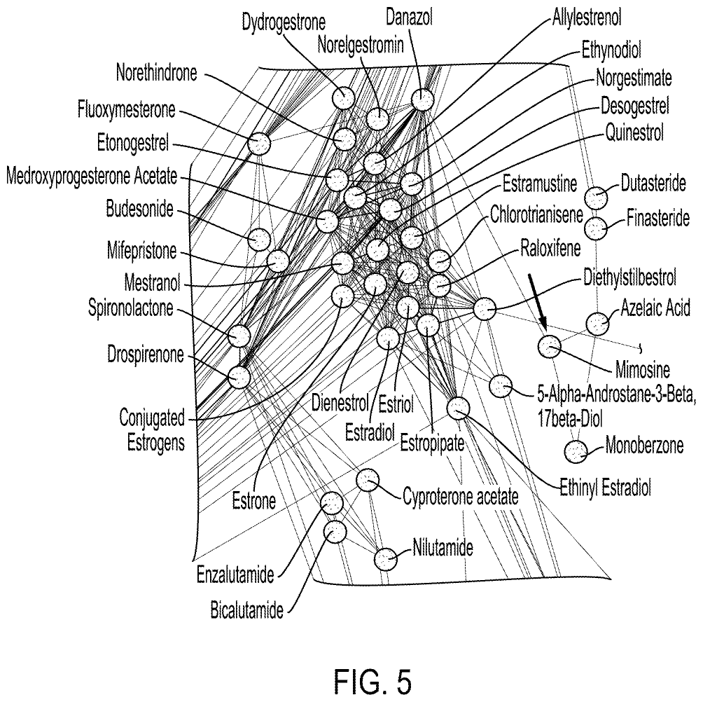

FIG. 5 depicts a zoomed DDSN detail of community C₆ (Drugs interfering with hormone-dependent cancers). The red arrow indicates the reconstructed drug repositioning: Mimosine—an experimental antineoplastic that inhibits DNA replication—also has effects in cancers affecting hormone-dependent organs.

### Reconstructed Repurposings as Anti-Inflammatory Drugs

According to the properties listed in DrugBank, the topological community C₃ includes drugs that exert anti-inflammatory effects via different mechanisms: non-steroidal anti-inflammatory drugs (e.g., Diclofenac, Ibuprofen, and Acetylsalicylic acid), the antirheumatic agent Auranofin, hypoglycemic drugs (e.g., Rosiglitazone, Troglitazone), and the antihypertensive drug Telmisartan. Moreover, the literature confirms that 28.57% of drugs within this community also present anti-inflammatory effects, even if they are not listed as anti-inflammatories in DrugBank. Here, the example of the versatile molecule of Fenofibrate is presented, which reduces the systemic inflammation independent of its lipid regulation effects, with cardiovascular benefits in high-risk and rheumatoid arthritis patients. Another illustrative example is that of Amiloride, which inhibits the activation of the dendritic cells and ameliorates the inflammation besides its diuretic effects, thus having benefits for hypertensive patients.

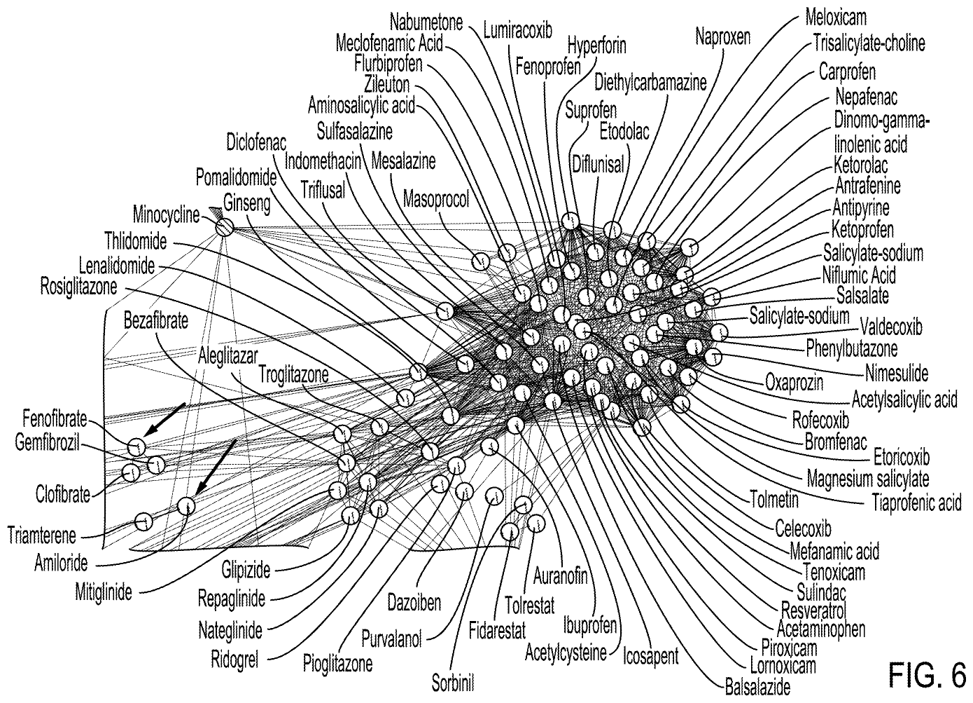

FIG. 6 depicts a zoomed DDSN detail of community C₃ (Anti-inflammatory drugs). The red arrows indicate the reconstructed drug repositionings as anti-inflammatory drugs: Fenofibrate (a lipid modifying drug) and Amiloride (a diuretic).

### Reconstructed Repurposings as Antifungal Drugs

The topological community C₂₅ includes 22 drugs. According to DrugBank, 13 out of these 22 drugs have antifungal properties, and 9 drugs act on the central nervous system (i.e., general anesthetics, sedative-hypnotics, and antiepileptics). DrugBank lists Isoflurane and Methoxyflurane as general anesthetic drugs. However, A. Giorgi et al. performed in vitro tests to investigate the antibacterial and antifungal effects of common anesthetic gases, and they found that Methoxyflurane and Isoflurane have excellent inhibitory effects on cultures of Klebsiella pneumoniae and Candida albicans. Using in vitro experiments, V. M. Barodka et al. also found that Isoflurane's liquid formulation has better anti-Candida activity than the antifungal Amphotericin B.

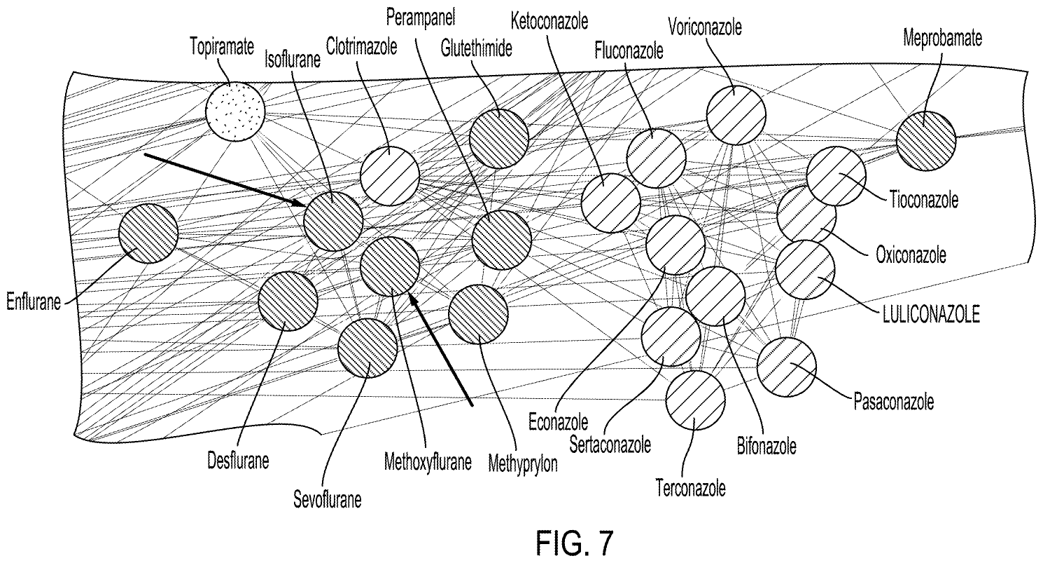

FIG. 7 depicts a zoomed DDSN detail of community C₂₅ (Antifungal agents). The red arrows indicate the reconstructed drug repositionings: Isoflurane and Methoxyflurane (known as general anesthetic drugs) also have antifungal effects.

### Repositioning Hints Prioritization

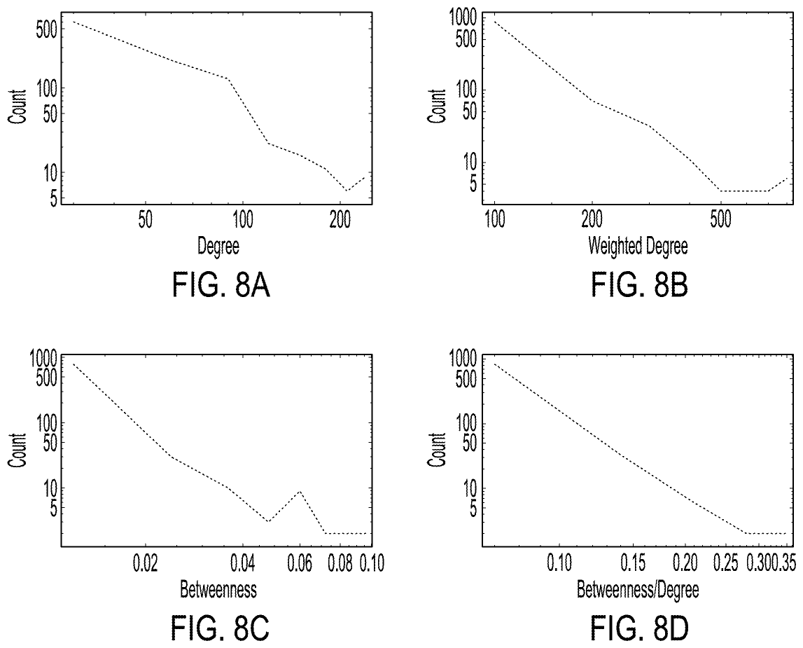

A high degree node can represent a drug with already documented multiple properties in our characterization of drug—drug similarity networks. Furthermore, a high betweenness (i.e., the ability to connect network communities) can indicate the drug's propensity for multiple pharmacological functions. By this logic, the high-betweenness, high-degree nodes may have reached their full repositioning potential, whereas the high betweenness, low degree nodes (characterized by high betweenness/degree value b/d) may indicate a significant repositioning potential. Predicting such high-value cases of degree d, weighted degree d_{w}, betweenness b, and betweenness/degree b/d can be difficult because the corresponding distributions are fat-tailed. Although all the estimated DDSN centralities follow a power-law distribution (see FIG. 8), the betweenness/degree b/d is the most stable parameter and, hence, the most reliable indicator of multiple drug properties.

FIG. 8 depicts power-law distributions of centrality parameters in the drug-drug similarity network (DDSN): (a) degree d; (b) weighted degree d_{w}; (c) betweenness b, and (d) betweenness/degree b/d. The distributions can be represented using 8 linearly spaced bins for each centrality. The fitting analysis using the Powerlaw package in Python indicates the following values for the distribution slope a and cutoff point xₘᵢₙ, respectively: 3.436 and 53 for d, 2.598 and 64 for d_{w}, 2.201 and 0.008 for b, 3.093 and 0.088 for b/d. The graphical representations of these centrality distributions show that the betweenness/degree b/d is the most stable parameter; therefore, it is the most reliable indicator of multiple drug properties.

To explore the capability of b/d to predict the multiple drug properties, the community structure of DDSN can be evaluated following a two-step approach. The relevant drug properties can be determined by generating network communities Cₓ with x=1̅,̅m̅ (m=26 in the DDSN). Then, using expert analysis, a dominant property can be assigned to each community. FIG. 3 illustrates the 26 DDSN communities as well as their dominant functionality. The dominant community property can be a pharmacological mechanism, a targeted disease, or a targeted organ. For instance, the community 1 (C₁) consists of antineoplastic drugs which act as mitotic inhibitors and DNA damaging agents; Community 13 (C₁₃) consists of cardiovascular drugs (antihypertensive, anti-arrhythmic, and anti-angina drugs), mostly beta-blockers.

In each cluster Cₓ, the top t drugs can be identified according to their b/d values. From these selected drugs, Bₓ^{t}⊂Cₓ, some may stand out by not sharing the community property or properties, and thus, can be repositioned as such. To this end, for x=1̅,̅ m̅ eliminated from Bₓ^{t} the drugs whose repurposings were already confirmed (i.e., performed by others and found in the recent literature), thus producing m=26 lists of repurposing hints yet to be confirmed by in silico, in vitro, and in vivo experiments, Bₓ^{h}=Bₓ^{t}\Bₓ^{c}. Table 3 presents the lists of Bₓ^{t} drugs for t=5 and x=26 (i.e., the top 5 b/d drugs in each community).

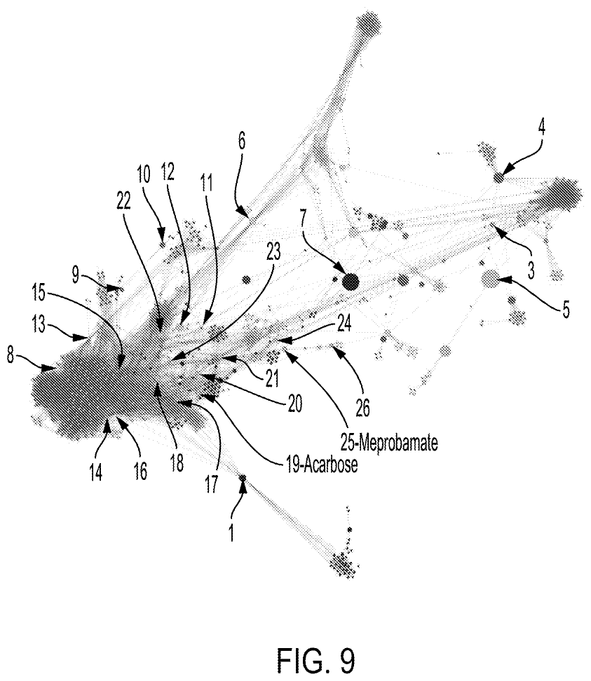

To facilitate the visual identification of the repositioning hints, in FIG. 9 the size of the nodes of the DDSN representation is shaped according to the magnitude of the b/d values.

By arrows, ] the top b/d nodes (i.e., drugs) are identified in their respective communities, by indicating their community id. Table 3 shows that Bₓ¹=Ø for all x except 19 and 25 (B₁₉¹={Acarbose} and B₁₉¹={Meprobamate}).

FIG. 9 depicts an example of a DDSN, based on drug-target interactions, where node sizes represent their b/d values. The arrows indicate the top b/d node in each community (for community 2, there is no top node because all drugs have

$$\frac{b}{d} = 0 ) .$$

The community index identifies each top b/d node, excepting Meprobamate (top b/d in community 25) and Acarbose (community 19), because these drugs (apparently) do not have their community's property; this indicates Meprobamate as antifungal (i.e., the property of community 25) and Acarbose as antiarrhythmic, anticonvulsant (i.e., the properties of community 19).

The high percentage of database and literature confirmations of the pharmacological properties predictions highlight the robustness of the repurposing method. Table 3 presents results indicating only a few unconfirmed drug properties (these repurposing hints ∈Bₓ^{h} are in bold).

TABLE 3
$Top 5 drugs ( {B}_{x}^{5} with x = \overline{1 , 26} ) according to their \frac{b}{d} values ,$
for each of the 26 DDSN communities/clusters (Cₓ). The properties of drugs
written in regular fonts match the properties of their respective communities
(according to the DrugBank). The properties of italicized drugs do not match
all their respective communities' properties, but the latest literature confirms
them (drugs in regular fonts and italics pertain to Bₓ^{h}). The properties of the
drugs written in bold correspond with new drug repositioning hints (i.e., the
Bₓ^{h} drugs). The positions marked with ‘—’ correspond to drugs with
$\frac{b}{d} = 0.$

| | Bₓ⁵ | | | | |
|---|---|---|---|---|---|
| Cₓ | 1 | 2 | 3 | 4 | 5 |
| 1 | Amsacrine | Colchicine | Podofilox | Lucanthone | Besifloxacin |
| 2 | — | — | — | — | — |
| 3 | Amiloride | Marimastat | Diclofenac | Thalidomide | Telmisartan |
| 4 | Minocycline | Framycetin | Amikacin | Doxycycline | — |
| | | | Tobramycin | Clomocycline | |
| | | | Netilmicin | Oxytetracycline | |
| 5 | Treprostinil | Iloprost | Captopril | Bimatoprost | Candoxatril |
| 6 | Progesterone | Mimosine | Fluticasone | Danazol | Spironolactone |
| | | | propionate | | |
| 7 | Vandetanib | Dalteparin | Dehydroepiandro- | Amlexanox | Atorvastatin |
| | | | sterone | | |
| 8 | Olopatadine | Terfenadine | Flunarizine | Astemizole | Epinastine |
| 9 | Succinylcholine | Carbachol | Decamethonium | Pilocarpine | Cevimeline |
| 10 | Nicotine | Melatonin | Amrinone | Dipyridamole | Naloxone |
| 11 | Quinine | Phenobarbital | Barbital | — | |
| | | Secobarbital | Hexobarbital | | |
| | | Pentobarbital | Aprobarbital - | | |
| 12 | Nimodipine | Adenosine | Drotaverine | Pentazocine | Loperamide |
| 13 | Ketotifen | Amiodarone | Sotalol | Bevantolol | Penbutolol |
| 14 | Disopyramide | Scopolamine | Ethopropazine | Paroxetine | Rocuronium |
| 15 | Minaprine | Amitriptyline | Agomelatine | Orphenadrine | Imipramine |
| 16 | Cocaine | Chloroprocaine | Procaine | Phenermine | Milnacipran |
| 17 | Epinephrine | 4-Methoxy- | Pseudoephedrine | Ephedra | Methamphetamine |
| | | amphetamine | | | |
| 18 | Ginkgo biloba | Captodiame | Cisapride | Bromocriptine | Carteolol |
| 19 | Acarbose | Lidocaine | Mexiletine | Etomidate | Flecainide |
| 20 | Phenelzine | Agmatine | Quinidine | Ephedrine | Amphetamine |
| | | | Propafenone | | |
| 21 | Zonisamide | Miconazole | Ethanol | Quinidine | — |
| 22 | Felodipine | Bepridil | Verapamil | Dextromethorphan | Amlodipine |
| 23 | Halothane | Halofantrine | Tramadol | Ibutilide | Tubocurarine |
| 24 | Thiamylal | Valproic Acid | Progabide | Bethanidine | Topiramate |
| 25 | Meprobamate | Enflurane | Tioconazole | Clotrimazole | Methoxyflurane |
| | | | | | Isoflurane |
| | | | | | Sevoflurane |
| 26 | Flunitrazepam | Eszopiclone | — | — | — |

The data indicates candidates for two top b/d drugs: Meprobamate, in the C₂₅ antifungal drugs community, and Acarbose, in the C₁₉ (Antiarrhythmics and Anticonvulsants) community. Both repositionings refer to properties currently unaccounted for. Meprobamate is a hypnotic, sedative, and mild muscle-relaxing drug, with no reported activities on the antifungal drug targets; thus, the antifungal activities of Meprobamate are not yet investigated in silico (with molecular docking), in vitro, or in vivo. Acarbose is a hypoglycemic drug, with no reported nor investigated antiarrhythmic and anticonvulsant properties. At the same time, repurposing hints may also be considered for drugs with high b/d, when the highest b/d values correspond to drugs already confirmed with the community property. For example, Azelaic acid has the highest b/d across not confirmed drugs in C₆.

### Repurposing Hints Testing

Molecular docking uses the target and ligand structures to predict the lead compound or repurpose drugs for different therapeutic purposes. The molecular docking tools predict the binding affinities, the preferred poses, and the ligand-receptor complex's interactions with minimum free energy. AutoDock 4.2.6 can be used, which consists of automated docking tools for predicting the binding of small ligands (i.e., drugs) to a macromolecule with an established 3D structure (i.e., target). The AutoDock semi-empirical free energy force field predicts the binding energy by considering complex energetic evaluations of bound and unbound forms of the ligand and the target, as well as an estimate of the conformational entropy lost upon binding.

As described herein, the predicted properties of repurposing hints can be verified by performing molecular docking not only for the hinted drugs but also for the reference drugs (typical drugs having the predicted property) and for some drugs with little probability of having the expected property. This way, the comparison between the interaction of the hinted drug with the biological targets—relevant for the tested property—and the interactions of the reference drugs with the same targets can be facilitated.

For example, the property ϕ can be considered as the anticancer effect with x=6 (corresponding to community C₆), and second ϕ as the antifungal effect with x=25 (community C₂₅). As such, the repurposing hints [symbol] ₕ^{ϕ}= [symbol] ₕ^{anticancer}={Azelaic acid} and [symbol] ₕ^{ϕ}= [symbol] ₕ^{antifungal}={Meprobamate} can be tested.

Accordingly, the anticancer reference drug can be defined from C₆ as [symbol] ₆^{anticancer}={Progesterone, Abiraterone}, no anticancer reference drug outside C₆ (i.e., [symbol] _{6̅}^{anticancer}=Ø), and two reference drugs with a low probability of anticancer effects [symbol] ₙ^{anticancer}={Fosinopril, Furosemide} (Fosinopril is an antihypertensive and Furosemide is a diuretic). Here, the interaction between the hinted and reference drugs with the targets can be tested from DrugBank associated with anticancer drugs in C₆, namely [symbol] ₆^{anticancer}={Progesterone receptor, Androgen receptor, Estrogen receptor beta, Steroid 17—alpha—hydroxylase/17, 20 lyase, Mineralocorticoid receptor, Estrogen receptor alpha}.

The antifungal references in C₂₅ as [symbol] ₂₅^{antifungal}={Clotrimazole, Oxiconazole}, and outside C₂₅ as [symbol] _{2̅5̅}^{antifungal}={Naftifine, Tolnaftate, Nystatin, Natamycin, Ciclopirox, Griseofulvin} can be evaluated. The reference drugs with little probability of having antifungal properties are [symbol] ₂₅^{antifungal}={Fosinopril, Furosemide}. The interactions between the hinted and reference drugs can be tested with DrugBank antifungal-related targets linked to drugs in C₂₅ and drugs not in C₂₅, respectively [symbol] _{2̅5̅}^{antifungal}={Lanosterol 14-alpha demethylase, Lanosterol synthase, Intermediate conductance calcium-activated potassium channel protein 4}, and [symbol] _{2̅5̅}^{antifungal}={Squalene monooxygenase, Ergosterol, Sodium/potassium-transporting ATPase subunit alpha, Tubulin}.

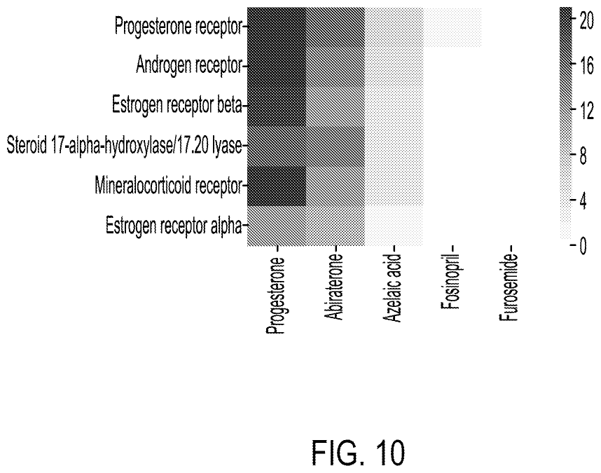

FIG. 10 depicts a summary of interactions resulted from the molecular docking analysis of the drug-target pairs generated with Equations (11)-(13) (Section 2, Section 2.3.1) for the hint [symbol] ₕ^{anticancer}={Azelaic acid}. For the hint and the reference drugs [symbol] ᵣ^{anticancer}, the interactions with the targets [symbol] ^{anticancer} are represented as the number of amino acids from the target interacting with the drug molecule (the maximum is 21). FIG. 10 depicts synthesis of interactions resulted from running molecular docking on the drug-target pairs for D_h\hat{ }ϕ=D_h\hat{ }anticancer={Azelaic Acid}. In the left part of the heatmap, we present the interactions between the relevant targets T\hat{ }anticancer={Progesterone receptor,Androgen receptor,Estrogen receptor beta, Steroid 17-alpha-hydroxylase/17,20 lyase,Mineralocorticoid receptor,Estrogen receptor alpha}, and the reference drugs D_r\hat{ }anticancer={Progesterone,Abiraterone}. In the right part of the heatmap, we present the interactions between the relevant targets T\hat{ }anticancer and the tested drugs D_t\hat{ }anticancer={Azelaic acid,Fosinopril,Furosemide}). We summarize the interactions with the targets T anticancer as the number of amino acids from the target interacting with the drug molecule (from 0 to the maximum number in our experiments, namely 21). The heatmap representation indicates interactions between d_h\hat{ }anticancer={Azelaic acid} and almost all the targets from T_6\hat{ }anticancer. For the drugs in D_n\hat{ }anticancer, namely Fosinopril and Furosemide, there is no interaction with the targets from T_6\hat{ }anticancer.

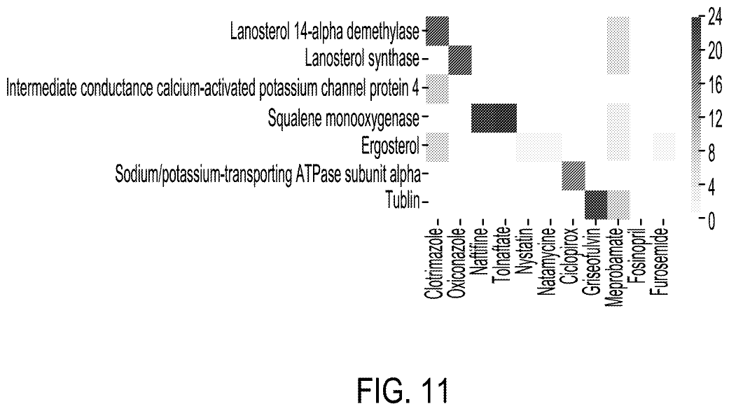

FIG. 11 depicts a summary of interactions resulted from the molecular docking analysis of the drug-target pairs generated with Equations (11)-(13) (see Section 2, Section 2.3.1), for the hint [symbol] ₕ^{ϕ}= [symbol] ₕ^{antifungal}={Meprobamate}. For the reference drugs [symbol] ᵣ^{antifungal}={Clotrimazole, Oxiconazole, Naftifine, Tolnaftate, Nystatin, Natamycin, Ciclopirox, Griseofulvin} the interaction is represented as the number of amino acids in the target interacting with the drug molecule (the maximum in the molecular docking experiments is 24). Because Ergosterol ∈ [symbol] _{2̅5̅}^{antifungal} has a steroidal chemical structure, instead of the number of amino acids, interaction strength is represented as the number of hydrophobic alkyl/alkyl interactions. For the tested drugs [symbol] ₜ^{antifungal}={Meprobamate, Fosinopril, Furosemide}, the interaction is represented as the number of amino acids from the target (or hydrophobic alkyl/alkyl interactions for Ergosterol) interacting in the same way with both the tested drug (∈ [symbol] ₜ^{antifungal}) and at least one reference drug (∈ [symbol] ₂₅^{antifungal}). The results confirm the interactions between dₕ^{antifungal} dantifungal (i.e., Meprobamate) and almost all the targets from both [symbol] ₂₅^{antifungal} and [symbol] _{2̅5̅}^{antifungal}. Conversely for the drugs in [symbol] ₙ^{antifungal}, there is no relevant interaction with any target from [symbol] ₂₅^{antifungal}∪ [symbol] _{2̅5̅}^{antifungal}. FIG. 11 depicts synthesis of interactions resulted from running molecular docking on the drug-target pairs for [symbol] ₕ^{ϕ}= [symbol] ₕ^{antifungal}={Meprobamate}. In the left part of the heatmap, we present the interactions between the relevant targets [symbol] ^{antifungal}={Lanosterol 14−alpha demethylase, Lanosterol synthase, Intermediate conductance calcium−activated potassium channel protein 4, Squalene monooxygenase, Ergosterol, Sodium/potassium−transporting ATPase subunit alpha, Tubulin} and the reference drugs [symbol] ᵣ^{antifungal}={Clotrimazole, Oxiconazole, Naftifine, Tolnaftate, Nystatin, Natamycin, Ciclopirox, Griseofulvin}. The reference drugs and targets pairs were tested that interact according to DrugBank. In the right part of the heatmap, the interactions between the relevant targets [symbol] ^{antifungal} and the tested drugs [symbol] ₜ^{antifungal}={Meprobamate, Fosinopril, Furosemide}) are presented. The interactions with the targets [symbol] ^{antifungal}T antifungal are presented as the number of amino acids from the target interacting with the drug molecule (from 0 to the maximum number in our experiments, namely 24). In the case of Ergosterol ∈ [symbol] _{2̅5̅}^{antifungal}, instead of the number of amino acids, the number of hydrophobic alkyl/alkyl interactions are counted because this target has a steroidal chemical structure. The heatmap representation indicates interactions between dₕ^{antifungal} (i.e., Meprobamate) and almost all the targets from both [symbol] ₂₅^{antifungal} and [symbol] _{2̅5̅}^{antifungal}. For the drugs in [symbol] ₙ^{antifungal}, there is no relevant interaction with any target from [symbol] ₂₅^{antifungal}∪ [symbol] _{2̅5̅}^{antifungal}.

After Autodock 4.2.6 and AutoDock Vina redocking, RMSD can be calculated in both cases. Low RMSD values were obtained (i.e., all of them are ≤1.016 Å), suggesting that the preliminary docking methodology is robust.

### Drug-Gene DDSN Repositioning Hints

A DDSN was generated using drug-gene interaction data from DrugBank 5.0.9, and clustered using modularity classes with λ=2.0 (as determined by optimizing λ over multiple candidate values for λ). Drug repositioning candidates were identified and confirmed against Drug bank 5.1.8 ATC codes: in particular, mepolizumab, listed as antineoplastic in DrugBank 5.0.9, was confirmed to also correspond to the respiratory system; naloxone, listed as opioid overdose antidote, was confirmed to also correspond to the nervous system; torasemide and quinetazone, listed as cardiovascular system in DrugBank 5.09, and methazolamide, acetazolamide, dorzolamide, and brinzolamide, listed as sensory organs, and zonisamide, listed as nervous system, also confirmed to also correspond to genitourinary system and sex hormone drugs.

A DDSN was generated using DrugBank 5.1.8, from which clusters were formed using λ=2.0. Drug repositioning candidates were confirmed against literature including pyridoxal phosphate, assigned alimentary tract and metabolism level 1 ATC code, also corresponding to nervous system; albendzaole, assigned antiparasitic products, insecticides, and repellants level 1 ATC code, also corresonding to ant-infectives for systemic use; methotrexate, assigned antineoplastic and immunomodulating agents level 1 ATC code, also corresponding to anti-infectives for systemic use; sysmvastatin, fluvastatin, lovastatin, and atorvastatin, assigned cardiovascular system level 1 ATC code, also corresponding to anti-infectives for systemic use; theophylline, assigned respiratory system level 1 ATC code, also corresponding to anticancer and immunomodulating properties; meloxicam, assigned musculo-skeletal system level 1 ATC code, also corresponding to anticancer and immunomodulating properties; cholecaliferol, ergocalciferol, and calcifediol, assigned alimentary tract and metabolism level 1 ATC code, also corresponding to antineoplastic and immunomodulating agents; chloroquine, assigned antiparasitic products, insectides, and repellents level 1 ATC code, also corresponding to antineoplastic and immunomodulating agents; mecasermin and mecasermin rinfabate, assigned systemic hormone preparations excluding sex hormones and insulins level 1 ATC code, also corresponding to alimentary tract and metabolism; and ornithine, assigned alimentary tract and metabolism level 1 ATC code, also corresponding to nervous system.

Drug repurposing as described herein can enable acceleration of drug discovery in sensitive areas of medicine, such as antibacterial resistance, complex life-threatening diseases (e.g., cancer), or rare diseases. Systems and methods in accordance with the present disclosure can implement a weighted drug-drug similarity network whose weights encode the existing known relationships among drugs (i.e., quantifies the number of biological targets shared by two drugs irrespective of the agonist or antagonist effect).

The ratio between node betweenness and node degree (i.e., a criterion of combined network metrics) can indicate the drug repositioning candidates better than considering simple network metrics (e.g., degree, weighted degree, betweenness). The power-law distributions in FIG. 8 can indicate that the DDSN is a complex system. Deciphering the emerging hidden higher-order functional interactions (i.e., interactions that span multiple orders of magnitude and involve multiple nodes) can be implemented by visualizing and analyzing the community structure in DDSN and determining the culprits (for such unknown functionalities) through combined network metrics criterion. The force-directed energy layout Force Atlas 2 can be used to generate network clusters of drugs because it emulates the emerging processes of a complex system. More precisely, the force-directed based network layouts can use micro-scale interactions (i.e., adjacent nodes attract and non-adjacent nodes repulse) to generate an emergent behavior at the macro-scale (i.e., topological clusters). Responsive to identifying communities, the combined network metrics criterion selects the drug repositioning most likely candidates. Specifically, the weighted drug-drug network analysis can encode not only information about how pairs of drugs interact with biological targets but also reveals the unknown functional relationship between drugs, such as the unknown effects on the activation/inhibition of a chemical pathway or cellular behavior. Underpinned by force-directed layout clustering can be used to analyze the fundamentally different structures represented by the drug-drug interaction networks (i.e., the DDIN interactome).

### Complex Network Perspective

Data incompleteness or scarcity can be a challenge for evaluating interactions. Systems and methods in accordance with the present disclosure can use a comprehensive database with a large and dense number of nodes/drugs and connections. b/d ranking can be used as a composite centrality because its distribution in DDSN is more stable than other centralities.

Azelaic acid (saturated dicarboxylic acid) and Meprobamate (carbamate derivative) can be selected as possible antineoplastic and antifungal from our repurposing hints list, respectively. Even so, one may find a posteriori confirmation clues for such repositioning hints. In the docking experiments, the two hints are not structurally similar to the respective reference drugs (i.e., Progesterone and Abiraterone for antineoplastic, and Clotrimazole, Oxiconazole, Naftifine, Tolnaftate, Nystatin, Natamycin, Ciclopirox, Griseofulvin for antifungal). Indeed, Progesterone and Abiraterone are steroid derivatives, Clotrimazole and Oxiconazole are imidazole derivatives, Ergosterol has a steroidal structure, Terbinafine and Naftifine are allylamine compounds, Griseofulvin is a 3-coumaranone derivative.

### Molecular Docking Perspective

Systems and methods in accordance with the present disclosure can use Molecular docking as an in silico simulation approach to drug discovery, which models the physical interaction between a ligand (i.e., small drug molecule) and a macromolecule (e.g., synthetic host macromolecule, biological target); it is also a valuable repurposing tool. The free energy values of the molecular interactions can be estimated with molecular docking to offer an approximation for the ligand's conformation and orientation into the protein cavity. DOCK can be used as a dedicated software tool in drug repurposing. Systems and methods in accordance with the present disclosure can enable molecular docking by providing drug repositioning hints (e.g., otherwise, the search space for drug repositionings can be exponentially big). For example, strong drug-target interaction hints can be generated such that large-scale drug-target interaction profiles can be generated. The molecular docking can be integrated with complex networks to determine new pharmacological properties by identifying new sets of biological targets on which the drug acts. The present solution can perform docking simulations including target baits (to reflect the limitations of false-positive and false-negative results), considering solvent effects, flexible docking, and comparing multiple docking tools.

The binding modes of Azelaic acid and Meprobamate can be compared to the other known reference drugs. The docking simulation results for the interaction between Azelaic acid and Steroid 17-alpha-hydroxylase/17,20 lyase, can be found to be highly similar to Progesterone and Abiraterone interactions with this target. Abiraterone is a potent 17-alpha-hydroxylase/17,20-lyase inhibitor used for the treatment of androgen-dependent prostate cancer. Therefore, discovering new drugs that inhibit this enzyme is a logical strategy. Because steroidal drugs—such as Abiraterone—have multiple steroid-related side effects, Hille et al. decided to synthesize non-steroidal compounds that mimic the natural 17-alpha-hydroxylase/17,20-lyase substrates (i.e., pregnenolone and progesterone). The docking simulation results presented herein are in line with evidence of the covalent bonding of Abiraterone to Steroid 17-alpha-hydroxylase/17,20 lyase (a cysteinato-heme enzyme from the cytochrome P450 superfamily). Precisely, Abiraterone forms a coordinate covalent bond of the pyridine nitrogen at C17 with this target's heme iron. Furthermore, the docking simulation of the interaction between Abiraterone and 17-alpha-hydroxylase/17,20-lyase confirms that Abiraterone establishes a hydrogen-bond between the —OH group and the target's Asn202; the results also confirm that amino acid residues of Phe114, Ile206, Leu209, Arg239, Gly301, and Val482 represent the hydrophobic environment for the reference Abiraterone. According to the docking simulation results, Azelaic acid does not establish a hydrogen bond with Asn202; however, not all the inhibitors tested by Chun-Zhi Ai et al. form a hydrogen bond with Asn202. (Instead, they bond to other amino acid residues than Abiraterone) Some hybrid aza-heterocycles compounds bound azelayl moiety through an amide bond that act as histone deacetylase inhibitors; this suggests the anticancer potential for three of their Azelaic acid derivatives in osteosarcoma among the five tumor cell lines tested.

Meprobamate has similar binding modes to that of Clotrimazole with Lanosterol 14 alpha-demethylase, Oxiconazole with Lanosterol synthase, and Griseofulvin with Tubulin. Indeed, we find the carbamate moiety in a wide range of drugs, such as Felbamate (anticonvulsant), Disulfiram (the treatment of chronic alcoholism), Rivastigmine (anti-dementia), Darunavir (antiviral for the treatment of HIV infections), or Physostigmine (antiglaucoma). Furthermore, carbamates are reversible acetylcholinesterase inhibitors that act as effective fungicides, insecticides, and herbicides in agriculture. Indeed, a recent reference reports the synthesis, in vitro, and in vivo antifungal evaluation of 36 novel threoninamide carbamate derivatives using the pharmacophore model.

Accordingly, the network-based computational drug repurposing method is robust, as it recovers a wide array of previous drug repositionings, such as demonstrated by employing the system using an older database, to validate the results with a new DrugBank version. In addition, systems and methods in accordance with the present disclosure can implement a testing prioritization method based on network centralities to make testing of the drug repositioning indicators more efficient. Validation of previously unaccounted drug properties using molecular docking is performed, demonstrating that the Azelaic acid represents a candidate for further in silico (e.g., molecular dynamics), in vitro, and in vivo investigations of its potential anticancer effects.

FIG. 12 depicts an example of a system 1200 to generate a DDSN and perform operations using the DDSN, using various processes and operations described herein and combinations thereof. The system 1200 can include one or more processors 1204 and memory 1208. The 1204 processor can be a general purpose or specific purpose processor, an application specific integrated circuit (ASIC), one or more field programmable gate arrays (FPGAs), a group of processing components, or other suitable processing components. The processor 1204 can execute computer code or instructions stored in memory or received from other computer readable media (e.g., CDROM, network storage, a remote server, etc.). The memory 1208 can include one or more devices (e.g., memory units, memory devices, storage devices, etc.) for storing data and/or computer code for completing and/or facilitating the various processes described in the present disclosure. Memory 1208 can include random access memory (RAM), read-only memory (ROM), hard drive storage, temporary storage, non-volatile memory, flash memory, optical memory, or any other suitable memory for storing software objects and/or computer instructions. Memory 1208 can include database components, object code components, script components, or any other type of information structure for supporting the various activities and information structures described in the present disclosure. Memory 1208 can be communicably connected to the processor 1204 and may include computer code for executing (e.g., by processor 1204) one or more processes described herein.

The system 1200 can include a communications circuit 1212, which can be used to transmit data to and from the processors 1204 and memory 1208 (along with databases from which data structures 1220 can be received). The communications circuit 1212 can include wired or wireless interfaces (e.g., jacks, antennas, transmitters, receivers, transceivers, wire terminals) for conducting data communications with various systems, devices, or networks. For example, the communications circuit 1212 can include an Ethernet card and port for sending and receiving data via an Ethernet-based communications network. The communications circuit 1212 can include a WiFi transceiver for communicating via a wireless communications network. The communications circuit 1212 can communicate via local area networks (e.g., a building LAN), wide area networks (e.g., the Internet, a cellular network), and/or conduct direct communications (e.g., NFC, Bluetooth). The communications circuit 1212 can conduct wired and/or wireless communications.

The system 1200 can include a user interface 1216. The user interface 1216 can receive user input and present information regarding operation of the system 1200. The user interface 536 may include one or more user input devices, such as buttons, dials, sliders, or keys, to receive input from a user. The user interface 1216 may include one or more display devices (e.g., OLED, LED, LCD, CRT displays), speakers, tactile feedback devices, or other output devices to provide information to a user. The user interface 1216 may execute a distributed application to receive user preference data.

The system 1200 can access at least one drug data structure 1220. For example, the system 1200 can receive data of drug data structures 1220 from various databases described herein, such as the DrugBank databases (e.g., various versions of DrugBank). The drug data structures 1220 can include one or more fields assigned one or more values, such as values relating to identifiers of drugs, identifiers of biological components (e.g., targets, genes, side effects (e.g., side effects associated with administering drugs)), characteristics of drugs (e.g., agonist, antagonist, or other relationships with biological components), and computational structures of drugs to be used for molecular docking or other computational operations on the drugs. The system 1200 can receive drug data structures 1220 or data thereof from one or more different sources, such as different databases, as well as from user interface 1216. The system 1200 can receive data regarding reference drugs expected to have particular characteristics, which can be used as control data for molecular docking or other operations for evaluating candidate drugs for repurposing.

The drug data structure 1220 can include an identifier 1224 of a drug, an identifier 1228 of a biological component, and a characteristic 1232 of a relationship between the drug and the biological component. For example, as depicted in FIG. 12, the drug data structure 1220 indicates that the Drug 1 has an agonist relationship with the Target 1. The characteristic 1232 can correspond to a function of the drug, such as a type of relationship the drug has with a particular biological component or a side effect resulting from interaction of the drug with a target, gene, or other biological component. The characteristic 1232 can be a type of an interaction, such as an inhibitor, agonist, antagonist, other/unkown, antibody, substrate, ligant, partial agonist, inducer, suppressor, binder, potentiator, modulator, activator, cofactor, degradation, positive allosteric modulator, incorporation into and destabilization, neutralizer, stimulator, binding, inactivator, inverse agonist, blocker, chaperone, inhibition of synthesis, antisense oligonucleotide, gene replacement, or regulator.

The system 1200 can generate at least one network 1236 using the drug data structures 1220. The network 1236 can stored by the system 1200 as a data structure. The network 1236 can include a plurality of nodes 1240 and a plurality of edges 1244. Each node 1240 can correspond to a drug identified from the drug data structures 1220.

The system 1200 can generate the edges 1244 using the information represented by the characteristics 1232. For example, the system 1200 can assign a first edge 1244 between a first node 1240 of a first drug and a second node 1240 of a second drug responsive to identifying at least one same characteristic of a relationship between the first drug and at least one first biological component and the second drug and the at least one first biological component. For example, if the first drug and the second drug each have an agonist relationship with each of two targets, the system 1200 can assign the first edge 1244 between the nodes 1240 of the first and second drug. The system 1200 can assign a weight to the first edge 1244 based on the same characteristics, such as assigning a weight of 4 to the first edge 1244 based on the first and second drug each having agonist relationships with the two targets. The system 1200 can normalize the weights assigned to the edges 1244, such as by dividing the sums of same relationships by a total number of edges 1244 (or total sum of all same relationships), among other normalization operations.

The system 1200 can generate subsets 1248 of nodes 1240 from amongst the nodes 1240 of the network 1236. For example, the system 1200 can generate the subsets 1248 to facilitate identifying similar drugs in order to detect repurposing or repositioning hints for drugs, such as where the network 1236 includes several drugs in a particular subset 1248, but not all of the drugs of the subset 1248 have previously been assigned a particular characteristic (which can be the same as the characteristic of the relationships the drugs are assigned with respect to common biological components, or can be an additional or alternative characteristic or classification assigned to the subset 1248, such as based on expert analysis or other labels as described herein), to allow the particular characteristic to be assigned to the drug(s) not having the particular characteristic assigned. For example, the system 1200 can assign, to a particular subset 1248 of nodes 1240, a particular characteristic (e.g., label), such as being an antifungal subset based on labeling received from user interface 1216, based on some of the nodes 1240 being assigned an antifungal label in the DrugBank databases, literature, or other data sources, or various combinations thereof; this characteristic can then be assigned to one or more nodes 1240 of the subset 1248 not previously assigned the antifungal label. The system 1200 can generate a histogram of characteristics (e.g., labels) for one or more subsets 1248, such as to assign the characteristic having the highest count for each respective subset 1248 of the one or more subset 1248 to the respective subset 1248.

For example, the system 1200 can generate subsets 1248 (e.g., clusters, communities) of nodes 1240 based on at least one of a modularity of at least one node 1240 or an energy of at least one node 1240. For example, ensemble methods, e.g. voting, averaging, weighted averaging methods, can be used to generate subsets 1248 from multiple types of processes, and combined or compared to generate drug repositioning lists. The modularity can indicate an edge density of a particular subset 1248 with respect to edge density between subsets 1248. The system 1200 can determine the modularity based on a number of edges in a particular subset 1248 relative to an expected number of edges in the particular subset 1248. The system 1200 can generate the subsets 1248 in a recursive process, such as a binary process in which two candidate subsets 1248 are determined from the network 1236, and then modified until a modularity of the subsets 1248 satisfies a target value (e.g., maximizing modularity). The binary process can be recursively applied to each subset until a total modularity of all subsets 1248 meets an end condition, such as the total modularity no longer increasing. For example, the subsets 1248 can be generated using Equations 1-4 described herein. In some implementations, the subsets 1248 are generated by assigning a cluster to each node 1240, and then moving nodes 1240 to different clusters responsive to determining that the move generates an increase in modularity, where a change in modularity is defined as:

$$Δ M = [ \frac{{K}_{{C}_{j}}^{*} + {K}_{i}^{{C}_{j}}}{2 a} - {( \frac{{K}_{{C}_{j}} + {K}_{i}}{2 a} )}^{2} ] - [ \frac{{K}_{{C}_{j}}^{*}}{2 a} - {( \frac{{K}_{{C}_{j}}}{2 a} )}^{2} - {( \frac{{K}_{i}}{2 a} )}^{2} ] \qquad ( 15 )$$

The system 1200 can determine the energy by arranging the nodes 1240 in a space, such as a two-dimensional space, and determining attraction forces between adjacent nodes and repulsion forces between non-adjacent nodes. For example, the system 1200 can arrange the nodes 1240 and apply an energy model force-directed layout to the nodes 1240 (e.g., using Equations 5 and 6) to adjust the positions of the nodes 1240 until the energy of the network 1236 satisfy an energy condition, such as a minimum energy threshold or to minimize the energy. The system 1200 can identify subsets 1248 (e.g., clusters or communities) from the arranged nodes 1240 based on identifying regions in the network 1236 having a density of edges 1244 greater than an average density of edges 1244 of the network 1236.

The system 1200 can identify characteristics to assign to one or more subsets 1248, as noted above, based on at least one of receiving labels to assign to subsets 1248, identifying labels of drugs in the subsets 1248, or various combinations thereof. For example, for a particular subset 1248, the system 1200 can identify characteristics assigned to the drugs of the subset 1248, and determine that a particular characteristic of the identified characteristics is assigned to at least a threshold amount of drugs of the subset 1248 in order to assign the particular characteristic to at least one drug of the subset 1248 to which the particular characteristic is not assigned. For example, the threshold amount can be fifty percent of the drugs in the subset 1248. The threshold amount can be adjusted based on validation of the repurposing. The system 1200 can store an association between the particular characteristic and at least one of the at least one drug or the node(s) 1240 of the at least one drug, in order to indicate that the at least one drug is a candidate for repurposing based on the particular characteristic. For example, if at least half of the drugs of a particular subset 1248 are antifungal drugs, the system 1200 can indicate, using the association, that one or more remaining drugs of the particular subset 1248 are candidates to repurpose for antifungal purposes.

The system 1200 can identify, from the subsets 1248, one or more candidate drugs for repurposing (e.g., repositioning). For example, the system 1200 can select a particular subset 1248, and identify one or more drugs of the subset 1248 (e.g. drugs associated with nodes 1240 of the subset 1248) to which the particular characteristic of the subset 1248 was not assigned, and provide the identified drugs for repurposing. The system 1200 can present output indicative of the identified drugs (e.g., using user interface and/or communications electronics). The system 1200 can perform various computational operations to evaluate the repurposing, such as by providing one or more of the identified drugs to a molecular docking operation with a target or other biological component associated with the particular characteristic.

The system 1200 can prioritize the identified drugs for repurposing, reducing resource demands, such as computational demands for molecular docking. For example, the system 1200 can evaluate a centrality of the one more identified drugs, assign a priority based on the centrality (e.g., higher priority for higher centrality), and select a subset of the identified drugs for repurposing. The system 1200 can determine the centrality based on a ratio of betweenness and degree of the nodes 1240 of the identified drugs (e.g., using Equations 7-10 described herein).

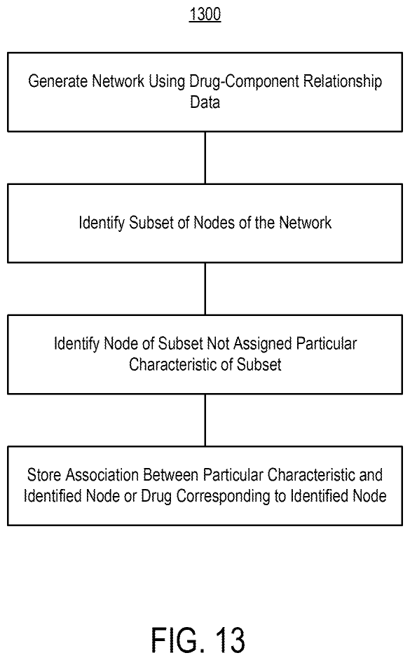

FIG. 13 depicts an example of a method 1300 of generating a DDSN and using the DDSN to generate drug repurposing candidates. The method 1300 can be performed using various systems, devices, and operations described herein, including but not limited to the system 1200. Various aspects of the method 1300 can be perform in parallel or in series, or various combinations thereof. The method 1300 can be performed responsive to receiving a request to perform one or more of generating a DDSN, updating the DDSN, identifying candidate drugs for repurposing using the DDSN, validating candidate drugs for repurposing, comparing drug repurposings from various sources (e.g., drug databases, literature, expert labeling), or various combinations thereof.

At 1305, a network is generated using characteristics of relationships between drugs and biological components. The network can be a DDSN. The network can be generated to include nodes corresponding to drugs, and edges between nodes that represent common or identical types of relationships between drugs and respective biological components, such as targets, genes, or side effects. For example, an edge can be assigned to connect two nodes based on the drugs of the two nodes both having the same of agonist or antagonist relationships with the same biological component; the edges can be weighted based on the number of such same relationships. As such, the edge can be generated based on (1) at least one first characteristic of the plurality of characteristics corresponding to the respective first drug and at least one first biological component of the plurality of targets and (2) at least one second characteristic of the plurality of characteristics corresponding to the respective second drug and the at least one first biological component. The edge can be weighted using a number of same characteristics amongst the at least one first characteristic and the at least one second characteristic with respect to the at least one first target. Generating the network can include generating a plurality of subsets of the network, such as clusters or communities, based on one or more parameters of the nodes and edges of the network, such as modularity or energy.

At 1310, a subset that includes at least three nodes is identified. For example, the subset can be a cluster or community, and can include a first identified node, a second identified node, and a third identified node. The subset can have a particular characteristic (e.g., anti-cancer, anti-fungal, etc.) that corresponds to the first and second identified nodes, while the third node is not assigned the particular characteristic. The particular characteristic can be accessed from the data structures having the data used to generate the network, expert labels, user input, or various combinations thereof. The subset can be of a plurality of subsets (e.g., clusters or communities) of the network, which can be generated by determining parameters of the network and nodes of the network such as modularity, energy (e.g., minimizing energy), or combinations thereof. The subset can be identified based on selecting the particular characteristic assigned to the subset.

At 1315, the particular characteristic is identified. For example, the particular characteristic can be identified responsive to a request to identify candidate drugs for repurposing for the particular characteristic (e.g., to select the subset associated with the particular characteristic and candidate drugs from the subset).

At 1320, an association can be stored between the particular characteristic and at least one of the third identified node and the drug corresponding to the third identified node. The association can indicate that the drug corresponding to the third identified node is a candidate drug for repurposing for the particular characteristic (e.g., even if databases such as the DrugBank or literature have not yet indicated that drug has the particular characteristic or has been verified to be able to perform a function associated with the particular characteristic). The association can be evaluated by applying the drug of the third identified node as input to an in silico validation operation, such as molecular docking, and comparing an output of the molecular docking operation with an expected output corresponding to the particular characteristic. Particular candidate drugs can be prioritized for evaluation based on properties of the nodes of the candidate drugs, such as centrality, to reduce computational demands for evaluating the repurposing of the candidate drugs. For example, the subset can include a plurality of third identified drugs that are not assigned the particular characteristic; the plurality of third identified drugs can be prioritized based on centrality (e.g., betweenness to degree ratio), and less than all of the third identified drugs having the highest priority can be selected for molecular docking or other in silico evaluation operations.

As utilized herein, the terms “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

The term “coupled,” as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. Such members may be coupled mechanically, electrically, and/or fluidly.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below,” etc.) are merely used to describe the orientation of various elements in the FIGURES. It should be noted that the orientation of various elements may differ according to other exemplary embodiments, and that such variations are intended to be encompassed by the present disclosure.

The hardware and data processing components used to implement the various processes, operations, illustrative logics, logical blocks, modules and circuits described in connection with the embodiments disclosed herein may be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device, etc.) may include one or more devices (e.g., RAM, ROM, Flash memory, hard disk storage, etc.) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit and/or the processor) the one or more processes described herein.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It is important to note that the construction and arrangement of the fluid control systems and methods of fluid control as shown in the various exemplary embodiments is illustrative only. Additionally, any element disclosed in one embodiment may be incorporated or utilized with any other embodiment disclosed herein. Although only one example of an element from one embodiment that can be incorporated or utilized in another embodiment has been described above, it should be appreciated that other elements of the various embodiments may be incorporated or utilized with any of the other embodiments disclosed herein.

### LIST OF REFERENCES

1. Dickson, M.; Gagnon, J. P. The cost of new drug discovery and development. Discov. Med. 2009, 4, 172-179.
2. Chen, X. Q.; Antman, M. D.; Gesenberg, C.; Gudmundsson, O. S. Discovery pharmaceutics—Challenges and opportunities. Aaps J. 2006, 8, E402E408. [CrossRef] [PubMed]
3. Mullard, A. 2016 FDA drug approvals. Nat. Rev. Drug Discov. 2017, 16, 73-76. [CrossRef] [PubMed]
4. Graul, A.; Pina, P.; Cruces, E.; Stringer, M. The year's new drugs & biologics 2016: Part I. Drugs Today 2017, 53, 27. [PubMed]
5. Pammolli, F.; Magazzini, L.; Riccaboni, M. The productivity crisis in pharmaceutical R&D. Nat. Rev. Drug Discov. 2011, 10, 428-438.
6. Drug Approvals and Databases. Available online: https://www.fda.gov/drugs/drug-approvals-and-databases/resources-information-approved-drugs (accessed on 15 Jan. 2019).
7. Csermely, P.; Korcsmáros, T.; Kiss, H. J.; London, G.; Nussinov, R. Structure and dynamics of molecular networks: A novel paradigm of drug discovery: A comprehensive review. Pharmacol. Ther. 2013, 138, 333-408. [CrossRef] [PubMed]
8. Pushpakom, S.; Iorio, F.; Eyers, P. A.; Escott, K. J.; Hopper, S.; Wells, A.; Doig, A.; Guilliams, T.; Latimer, J.; McNamee, C.; et al. Drug repurposing: Progress, challenges and recommendations. Nat. Rev. Drug Discov. 2019, 18, 41. [CrossRef]
9. Munos, B. Lessons from 60 years of pharmaceutical innovation. Nat. Rev. Drug Discov. 2009, 8, 959-968. [CrossRef]
10. Shaughnessy, A. F. Old drugs, new tricks. BMJ 2011, 342, d741. [CrossRef]
11. Li, J.; Zheng, S.; Chen, B.; Butte, A. J.; Swamidass, S. J.; Lu, Z. A survey of current trends in computational drug repositioning. Brief Bioinform. 2015, 17, 2-12. [CrossRef]
12. Lotfi Shahreza, M.; Ghadiri, N.; Mousavi, S. R.; Varshosaz, J.; Green, J. R. A review of network-based approaches to drug repositioning. Brief Bioinform. 2017, 19, 878-892. [CrossRef] [PubMed]
13. Nugent, T.; Plachouras, V.; Leidner, J. L. Computational drug repositioning based on side-effects mined from social media. Peerj Comput. Sci. 2016, 2, e46. [CrossRef]
14. Zhao, M.; Yang, C. C. Mining Online Heterogeneous Healthcare Networks for Drug Repositioning. In Proceedings of the Healthcare Informatics (ICHI), 2016 IEEE International Conference, Chicago, IL, USA, 4-7 Oct. 2016; pp. 106-112.
15. Shameer, K.; Readhead, B.; T Dudley, J. Computational and experimental advances in drug repositioning for accelerated therapeutic stratification. Curr. Top. Med. Chem. 2015, 15, 5-20. [CrossRef] [PubMed]
16. Yildirim, M. A.; Goh, K. I.; Cusick, M. E.; Barabási, A. L.; Vidal, M. Drug-target network. Nat. Biotechnol. 2007, 25, 1119-1126. [CrossRef] [PubMed]
17. Mei, J. P.; Kwoh, C. K.; Yang, P.; Li, X. L.; Zheng, J. Drug-target interaction prediction by learning from local information and neighbors. Bioinformatics 2012, 29, 238-245. [CrossRef]
18. Wang, W.; Yang, S.; Zhang, X.; Li, J. Drug repositioning by integrating target information through a heterogeneous network model. Bioinformatics 2014, 30, 2923-2930. [CrossRef]
19. Luo, Y.; Zhao, X.; Zhou, J.; Yang, J.; Zhang, Y.; Kuang, W.; Peng, J.; Chen, L.; Zeng, J. A Network Integration Approach for Drug-Target Interaction Prediction and Computational Drug Repositioning from Heterogeneous Information. Nat. Commun. 2017, 8, 573. [CrossRef]
20. Wu, Z.; Li, W.; Liu, G.; Tang, Y. Network-based methods for prediction of drug-target interactions. Front. Pharmacol. 2018, 9, 1134. [CrossRef]
21. Tanoli, Z.; Alam, Z.; Ianevski, A.; Wennerberg, K.; Vähä-Koskela, M.; Aittokallio, T. Interactive visual analysis of drug-target interaction networks using drug target profiler, with applications to precision medicine and drug repurposing. Brief Bioinform. 2018, 21, 211-220. [CrossRef]
22. Iorio, F.; Bosotti, R.; Scacheri, E.; Belcastro, V.; Mithbaokar, P.; Ferriero, R.; Murino, L.; Tagliaferri, R.; Brunetti-Pierri, N.; Isacchi, A.; et al. Discovery of drug mode of action and drug repositioning from transcriptional responses. Proc. Natl. Acad. Sci. USA 2010, 107, 14621-14626. [CrossRef]
23. Iorio, F.; Rittman, T.; Ge, H.; Menden, M.; Saez-Rodriguez, J. Transcriptional data: A new gateway to drug repositioning? Drug Discov. Today 2013, 18, 350-357. [CrossRef]
24. Cheng, F.; Kovács, I. A.; Barabási, A. L. Network-based prediction of drug combinations. Nat. Commun. 2019, 10, 1197. [CrossRef]
25. Cheng, F.; Desai, R. J.; Handy, D. E.; Wang, R.; Schneeweiss, S.; Barabási, A. L.; Loscalzo, J. Network-based approach to prediction and population-based validation of in silico drug repurposing. Nat. Commun. 2018, 9, 2691. [CrossRef]
26. Nguyen, T.; Le, H.; Venkatesh, S. GraphDTA: Prediction of drug-target binding affinity using graph convolutional networks. BioRxiv 2019, 684662. [CrossRef]
27. Zitnik, M.; Agrawal, M.; Leskovec, J. Modeling polypharmacy side effects with graph convolutional networks. Bioinformatics 2018, 34, i457-i466. [CrossRef]
28. Mayr, A.; Klambauer, G.; Unterthiner, T.; Steijaert, M.; Wegner, J. K.; Ceulemans, H.; Clevert, D. A.; Hochreiter, S. Large-scale comparison of machine learning methods for drug target prediction on ChEMBL. Chem. Sci. 2018, 9, 5441-5451. [CrossRef]
29. Lo, Y. C.; Rensi, S. E.; Torng, W.; Altman, R. B. Machine learning in chemoinformatics and drug discovery. Drug Discov. Today 2018, 23, 1538-1546. [CrossRef] [PubMed]
30. Liu, Z.; Fang, H.; Reagan, K.; Xu, X.; Mendrick, D. L.; Slikker, W.; Tong, W. In silico drug repositioning—what we need to know. Drug Discov. Today 2013, 18, 110-115. [CrossRef]
31. Kunimoto, R.; Bajorath, J. Design of a tripartite network for the prediction of drug targets. J.-Comput.-Aided Mol. Des. 2018, 32, 321-330. [CrossRef]
32. Hopkins, A. L. Network pharmacology: The next paradigm in drug discovery. Nat. Chem. Biol. 2008, 4, 682-690. [CrossRef]
33. Wishart, D. S.; Knox, C.; Guo, A. C.; Cheng, D.; Shrivastava, S.; Tzur, D.; Gautam, B.; Hassanali, M. DrugBank: A knowledgebase for drugs, drug actions and drug targets. Nucleic Acids Res. 2007, 36, D901-D906. [CrossRef] [PubMed]
34. Jacomy, M.; Venturini, T.; Heymann, S.; Bastian, M. ForceAtlas2, a continuous graph layout algorithm for handy network visualization designed for the Gephi software. PLoS ONE 2014, 9, e98679. [CrossRef] [PubMed]
35. Girvan, M.; Newman, M. E. Community structure in social and biological networks. Proc. Natl. Acad. Sci. USA 2002, 99, 7821-7826. [CrossRef]
36. Ferreira, L. G.; Dos Santos, R. N.; Oliva, G.; Andricopulo, A. D. Molecular docking and structure-based drug design strategies. Molecules 2015, 20, 13384-13421. [CrossRef]
37. Wishart, D. S.; Feunang, Y. D.; Guo, A. C.; Lo, E. J.; Marcu, A.; Grant, J. R.; Sajed, T.; Johnson, D.; Li, C.; Sayeeda, Z.; et al. DrugBank 5.0: A major update to the DrugBank database for 2018. Nucleic Acids Res. 2017, 46, D1074-D1082. [CrossRef]
38. Bastian, M.; Heymann, S.; Jacomy, M. Gephi: An open source software for exploring and manipulating networks. Icwsm 2009, 8, 361-362.
39. Newman, M. E. Modularity and community structure in networks. Proc. Natl. Acad. Sci. USA 2006, 103, 8577-8582. [CrossRef]
40. Newman, M. E. Equivalence between modularity optimization and maximum likelihood methods for community detection. Phys. Rev. E 2016, 94, 052315. [CrossRef]
41. Noack, A. Modularity clustering is force-directed layout. Phys. Rev. E 2009, 79, 026102. [CrossRef]
42. Barabási, A. L. Network Science; Cambridge University Press: Cambridge, UK, 2016.
43. Topirceanu, A.; Udrescu, M.; Marculescu, R. Weighted betweenness preferential attachment: A new mechanism explaining social network formation and evolution. Sci. Rep. 2018, 8, 10871. [CrossRef]
44. Protain Data Bank. Available online: http://www.rcsb.org/pdb/home/home.do (accessed on 25 May 2020).
45. Zhang Lab. Available online: https://zhanglab.ccmb.med.umich.edu/ModRefiner/ (accessed on 25 May 2020).
46. Morris, G. M.; Huey, R.; Lindstrom, W.; Sanner, M. F.; Belew, R. K.; Goodsell, D. S.; Olson, A. J. AutoDock4 and AutoDockTools4: Automated docking with selective receptor flexibility. J. Comput. Chem. 2009, 30, 2785-2791. [CrossRef] [PubMed]
47. Grathwohl, W.; Wang, K. C.; Jacobsen, J. H.; Duvenaud, D.; Norouzi, M.; Swersky, K. Your Classifier is Secretly an Energy Based Model and You Should Treat it Like One. arXiv 2019, arXiv:1912.03263.
48. Udrescu, L.; Sbârcea, L.; Topîrceanu, A.; Iovanovici, A.; Kurunczi, L.; Bogdan, P.; Udrescu, M. Clustering drug-drug interaction networks with energy model layouts: Community analysis and drug repurposing. Sci. Rep. 2016, 6, 32745. [CrossRef] [PubMed]
49. Cho, J. H.; Joo, Y. H.; Shin, E. Y.; Park, E. J.; Kim, M. S. Anticancer effects of colchicine on hypopharyngeal cancer. Anticancer Res. 2017, 37, 6269-6280.
50. Shen, M.; Zhang, Y.; Saba, N.; Austin, C. P.; Wiestner, A.; Auld, D. S. Identification of therapeutic candidates for chronic lymphocytic leukemia from a library of approved drugs. PLoS ONE 2013, 8, e75252. [CrossRef]
51. Melo, S.; Villanueva, A.; Moutinho, C.; Davalos, V.; Spizzo, R.; Ivan, C.; Rossi, S.; Setien, F.; Casanovas, O.; Simo-Riudalbas, L.; et al. Small molecule enoxacin is a cancer-specific growth inhibitor that acts by enhancing TAR RNA-binding protein 2-mediated microRNA processing. Proc. Natl. Acad. Sci. USA 2011, 108, 4394-4399. [CrossRef]
52. Yadav, V.; Varshney, P.; Sultana, S.; Yadav, J.; Saini, N. Moxifloxacin and ciprofloxacin induces S-phase arrest and augments apoptotic effects of cisplatin in human pancreatic cancer cells via ERK activation. BMC Cancer 2015, 15, 581. [CrossRef]
53. Fabian, I.; Reuveni, D.; Levitov, A.; Halperin, D.; Priel, E.; Shalit, I. Moxifloxacin enhances antiproliferative and apoptotic effects of etoposide but inhibits its proinflammatory effects in THP-1 and Jurkat cells. Br. J. Cancer 2006, 95, 1038-1046. [CrossRef]54. Yadav, V.; Sultana, S.; Yadav, J.; Saini, N. Gatifloxacin induces S and G2-phase cell cycle arrest in pancreatic cancer cells via p21/p27/p53. PLoS ONE 2012, 7, e47796. [CrossRef]
55. Collaborative Group on Hormonal Factors in Breast Cancer. Type and timing of menopausal hormone therapy and breast cancer risk: Individual participant meta-analysis of the worldwide epidemiological evidence. Lancet 2019, 394, 1159-1168. [CrossRef]
56. Ma, H.; Bernstein, L.; Pike, M. C.; Ursin, G. Reproductive factors and breast cancer risk according to joint estrogen and progesterone receptor status: A meta-analysis of epidemiological studies. Breast Cancer Res. 2006, 8, R43. [CrossRef] [PubMed]
57. Leo, J. C.; Wang, S. M.; Guo, C. H.; Aw, S. E.; Zhao, Y.; Li, J. M.; Hui, K. M.; Lin, V. C. Gene regulation profile reveals consistent anticancer properties of progesterone in hormone-independent breast cancer cells transfected with progesterone receptor. Int. J. Cancer 2005, 117, 561-568. [CrossRef] [PubMed]
58. Preet, R.; Mohapatra, P.; Mohanty, S.; Sahu, S. K.; Choudhuri, T.; Wyatt, M. D.; Kundu, C. N. Quinacrine has anticancer activity in breast cancer cells through inhibition of topoisomerase activity. Int. J. Cancer 2012, 130, 1660-1670. [CrossRef]
59. Yap, T. A.; Smith, A. D.; Ferraldeschi, R.; Al-Lazikani, B.; Workman, P.; De Bono, J. S. Drug discovery in advanced prostate cancer: Translating biology into therapy. Nat. Rev. Drug Discov. 2016, 15, 699. [CrossRef]
60. Chung, L. C.; Tsui, K. H.; Feng, T. H.; Lee, S. L.; Chang, P. L.; Juang, H. H. L-Mimosine blocks cell proliferation via upregulation of B-cell translocation gene 2 and N-myc downstream regulated gene 1 in prostate carcinoma cells. Am. J. Physiol.-Cell Physiol. 2012, 302, C676-C685. [CrossRef]
61. Belfort, R.; Berria, R.; Cornell, J.; Cusi, K. Fenofibrate reduces systemic inflammation markers independent of its effects on lipid and glucose metabolism in patients with the metabolic syndrome. J. Clin. Endocrinol. Metab. 2010, 95, 829-836. [CrossRef]
62. Goto, M. A comparative study of anti-inflammatory and antidyslipidemic effects of fenofibrate and statins on rheumatoid arthritis. Mod. Rheumatol. 2010, 20, 238-243. [CrossRef]
63. Barbaro, N. R.; Foss, J. D.; Kryshtal, D. O.; Tsyba, N.; Kumaresan, S.; Xiao, L.; Mernaugh, R. L.; Itani, H. A.; Loperena, R.; Chen, W.; et al. Dendritic cell amiloride-sensitive channels mediate sodium-induced inflammation and hypertension. Cell Rep. 2017, 21, 1009-1020. [CrossRef] [PubMed]
64. Giorgi, A.; Parodi, F.; Piacenza, G.; Mantellini, E.; Salio, M.; Cremonte, L.; Grosso, E. Antibacterial and antifungal activity of isoflurane and common anesthetic gases. Minerva Med. 1986, 77, 2007-2010.
65. Barodka, V. M.; Acheampong, E.; Powell, G.; Lobach, L.; Logan, D. A.; Parveen, Z.; Armstead, V.; Mukhtar, M. Antimicrobial effects of liquid anesthetic isoflurane on Candida albicans. J. Transl. Med. 2006, 4, 46. [CrossRef] [PubMed]
66. Clauset, A.; Shalizi, C. R.; Newman, M. E. Power-law distributions in empirical data. SIAM Rev. 2009, 51, 661-703. [CrossRef]
67. Alstott, J.; Bullmore, E.; Plenz, D. powerlaw: A Python package for analysis of heavy-tailed distributions. PLoS ONE 2014, 9, e85777. [CrossRef] [PubMed]
68. Nunes, R. R.; Fonseca, A. L. d.; Pinto, A. C. D. S.; Maia, E. H. B.; Silva, A. M. D.; Varotti, F. D. P.; Taranto, A. G. Brazilian malaria molecular targets (BraMMT): Selected receptors for virtual high-throughput screening experiments. Mem. Inst. Oswaldo Cruz 2019, 114. [CrossRef]
69. Udrescu, M.; Udrescu, L. A Drug Repurposing Method Based on Drug-Drug Interaction Networks and Using Energy Model Layouts. In Computational Methods for Drug Repurposing; Springer: Berlin/Heidelberg, Germany, 2019; pp. 185-201.
70. Mestres, J.; Gregori-Puigjane, E.; Valverde, S.; Sole, R. V. Data completeness—The Achilles heel of drug-target networks. Nat. Biotechnol. 2008, 26, 983-984. [CrossRef] [PubMed]
71. Borgatti, S. P.; Carley, K. M.; Krackhardt, D. On the robustness of centrality measures under conditions of imperfect data. Soc. Net. 2006, 28, 124-136. [CrossRef]
72. Iyer, S.; Killingback, T.; Sundaram, B.; Wang, Z. Attack robustness and centrality of complex networks. PLoS ONE 2013, 8, e59613. [CrossRef]
73. Breathnach, A. Azelaic acid: Potential as a general antitumoural agent. Med. Hypotheses 1999, 52, 221-226. [CrossRef]
74. Colovic, M. B.; Krstic, D. Z.; Lazarevic-Pasti, T. D.; Bondzic, A. M.; Vasic, V. M. Acetylcholinesterase inhibitors: Pharmacology and toxicology. Curr. Neuropharmacol. 2013, 11, 315-335. [CrossRef]
75. Udrescu, L.; Sbârcea, L.; Fulias, A.; Ledet, i, I.; Vlase, G.; Barvinschi, P.; Kurunczi, L. Physicochemical analysis and molecular modeling of the Fosinopril β-cyclodextrin inclusion complex. J. Spectrosc. 2014, 2014. [CrossRef]
76. Chittepu, V. C.; Kalhotra, P.; Osorio-Gallardo, T.; Gallardo-Velázquez, T.; Osorio-Revilla, G. Repurposing of FDA-approved NSAIDs for DPP-4 inhibition as an alternative for diabetes mellitus treatment: Computational and in vitro study. Pharmaceutics 2019, 11, 238. [CrossRef]
77. Ekins, S.; Mestres, J.; Testa, B. In silico pharmacology for drug discovery: Methods for virtual ligand screening and profiling. Br. J. Pharmacol. 2007, 152, 9-20. [CrossRef] [PubMed]
78. Ewing, T. J.; Makino, S.; Skillman, A. G.; Kuntz, I. D. DOCK 4.0: Search strategies for automated molecular docking of flexible molecule databases. J. Comput.-Aided Mol. Des. 2001, 15, 411-428. [CrossRef] [PubMed]
79. DesJarlais, R.; Seibel, G.; Kuntz, I.; Furth, P.; Alvarez, J.; De Montellano, P. O.; DeCamp, D.; Babe, L.; Craik, C. Structure-based design of nonpeptide inhibitors specific for the human immunodeficiency virus 1 protease. Proc. Natl. Acad. Sci. USA 1990, 87, 6644-6648. [CrossRef] [PubMed]
80. Cavalla, D.; Oerton, E.; Bender, A. Drug repurposing review. Ref. Modul. Chem. Mol. Sci. Chem. Eng. 2017. [CrossRef]
81. Vieira, T. F.; Sousa, S. F. Comparing AutoDock and Vina in Ligand/Decoy Discrimination for Virtual Screening. Appl. Sci. 2019, 9, 4538. [CrossRef]
82. Elokely, K. M.; Doerksen, R. J. Docking challenge: Protein sampling and molecular docking performance. J. Chem. Inf. Model. 2013, 53, 1934-1945. [CrossRef]
83. Maia, E. H.; Medaglia, L. R.; da Silva, A. M.; Taranto, A. G. Molecular Architect: A User-Friendly Workflow for Virtual Screening. ACS Omega 2020, 5, 6628-6640. [CrossRef]
84. Martin, Y. C.; Kofron, J. L.; Traphagen, L. M. Do structurally similar molecules have similar biological activity? J. Med. Chem. 2002, 45, 4350-4358. [CrossRef]
85. Yang, L.; Chen, J.; Shi, L.; Hudock, M. P.; Wang, K.; He, L. Identifying unexpected therapeutic targets via chemical-protein interactome. PLoS ONE 2010, 5, e9568. [CrossRef]
86. Simon, Z.; Peragovics, Á.; Vigh-Smeller, M.; Csukly, G.; Tombor, L.; Yang, Z.; Zahoránszky-Köhalmi, G.; Végner, L.; Jelinek, B.; Hári, P.; et al. Drug effect prediction by polypharmacology-based interaction profiling. J. Chem. Inf. Model. 2012, 52, 134-145. [CrossRef]
87. Haupt, V. J.; Schroeder, M. Old friends in new guise: Repositioning of known drugs with structural bioinformatics. Briefings Bioinform. 2011, 12, 312-326. [CrossRef] [PubMed]
88. Hille, U. E.; Hu, Q.; Vock, C.; Negri, M.; Bartels, M.; Müller-Vieira, U.; Lauterbach, T.; Hartmann, R. W. Novel CYP17 inhibitors: Synthesis, biological evaluation, structure—activity relationships and modelling of methoxy-and hydroxy-substituted methyleneimidazolyl biphenyls. Eur. J. Med. Chem. 2009, 44, 2765-2775. [CrossRef] [PubMed]
89. Avendaño, C.; Menendez, J. C. Medicinal Chemistry of Anticancer Drugs; Elsevier: Amsterdam, The Netherlands, 2015.
90. DeVore, N. M.; Scott, E. E. Structures of cytochrome P450 17A1 with prostate cancer drugs abiraterone and TOK-001. Nature 2012, 482, 116-119. [CrossRef] [PubMed]
91. Ai, C. Z.; Man, H. Z.; Saeed, Y.; Chen, D. C.; Wang, L. H.; Jiang, Y. Z. Computational insight into crucial binding features for metabolic specificity of cytochrome P450 17A1. Inform. Med. Unlocked 2019, 15, 100172. [CrossRef]
92. Micheletti, G.; Calonghi, N.; Farruggia, G.; Strocchi, E.; Palmacci, V.; Telese, D.; Bordoni, S.; Frisco, G.; Boga, C. Synthesis of Novel Structural Hybrids between Aza-Heterocycles and Azelaic Acid Moiety with a Specific Activity on Osteosarcoma Cells. Molecules 2020, 25, 404. [CrossRef]
93. Du, X. J.; Peng, X. J.; Zhao, R. Q.; Zhao, W. G.; Dong, W. L.; Liu, X. H. Design, synthesis and antifungal activity of threoninamide carbamate derivatives via pharmacophore model. J. Enzym. Inhib. Med. Chem. 2020, 35, 682-691. [CrossRef]

1. A method, comprising:
generating, by one or more processors using a plurality of characteristics of relationships between a plurality of drugs and a plurality of biological components, a network comprising a plurality of nodes and a plurality of edges, each edge of the plurality of edges connecting a respective first node of the plurality of nodes and a respective second node of the plurality of nodes, the respective first node corresponding to a respective first drug of the plurality of drugs, the respective second node corresponding to a respective second drug of the plurality of drugs, each edge generated based on (1) at least one first characteristic of the plurality of characteristics corresponding to the respective first drug and at least one first biological component of the plurality of targets and (2) at least one second characteristic of the plurality of characteristics corresponding to the respective second drug and the at least one first biological component;
identifying, by the one or more processors, a subset comprising at least a first identified node, a second identified node, and a third identified node of the plurality of nodes;
identifying, by the one or more processors, a particular characteristic of the subset, the drug of the third node not assigned the particular characteristic; and
storing, by the one or more processors, an association between the particular characteristic and at least one of the third identified node and the drug of the third identified node.

2. The method of claim 1, further comprising evaluating, by the one or more processors, the association by:
applying, as input to a molecular docking operation, the drug of the third identified node; and
comparing an output of the molecular docking operation with an expected output corresponding to the particular characteristic.

3. The method of claim 1, wherein generating the first edge comprises assigning a weight to the first edge corresponding to a number of same characteristics amongst the at least one first characteristic and the at least one second characteristic with respect to the at least one first target.

4. The method of claim 1, further comprising generating a plurality of clusters from the plurality of nodes of the network, the subset corresponding to a cluster of the plurality of clusters.

5. The method of claim 4, wherein generating the plurality of clusters comprises evaluating a modularity of the network, the modularity corresponding to an amount of the plurality of edges of one or more clusters of the plurality of clusters relative to an expected amount of edges.

6. The method of claim 4, wherein generating the plurality of clusters comprises evaluating an energy of the network, the energy corresponding to distances between adjacent nodes of the network and distances between non-adjacent nodes of the network.

7. The method of claim 4, further comprising determining a centrality of at least one node of the plurality of nodes, the centrality based on a degree of the at least one node and a betweenness of the at least one node, the degree corresponding to a number of edges connected with the node, the betweenness corresponding to a number of paths in the network through the at least one node.

8. The method of claim 7, further comprising prioritizing, by the one or more processors, validation of the association between the particular characteristic and the third drug based on the centrality of the third node.

9. The method of claim 1, further comprising validating the association between the particular characteristic and the third drug by performing molecular docking using the third drug, a first reference drug having the particular characteristic, and a second reference drug for which a probability of having the particular characteristic is less than a threshold value.

10. (canceled)

11. The method of claim 1, wherein the particular characteristic comprises at least one of a pharmacological mechanism, a targeted disease, or a targeted organ.

12. The method of claim 11, wherein the association indicates a potential repurposing of the third drug for the particular characteristic.

13. The method of claim 1, wherein the plurality of biological elements comprise at least one of a drug target, a gene, or a side effect of drug administration.

14. The method of claim 1, wherein each characteristic of the at least one first characteristic is one of an inhibitor, agonist, antagonist, other/unkown, antibody, substrate, ligant, partial agonist, inducer, suppressor, binder, potentiator, modulator, activator, cofactor, degradation, positive allosteric modulator, incorporation into and destabilization, neutralizer, stimulator, binding, inactivator, inverse agonist, blocker, chaperone, inhibition of synthesis, antisense oligonucleotide, gene replacement, or regulator.

15. A system, comprising:
one or more processors configured to:
generate, using a plurality of characteristics of relationships between a plurality of drugs and a plurality of biological components, a network comprising a plurality of nodes and a plurality of edges, each edge of the plurality of edges connecting a respective first node of the plurality of nodes and a respective second node of the plurality of nodes, the respective first node corresponding to a respective first drug of the plurality of drugs, the respective second node corresponding to a respective second drug of the plurality of drugs, each edge generated based on (1) at least one first characteristic of the plurality of characteristics corresponding to the respective first drug and at least one first biological component of the plurality of targets and (2) at least one second characteristic of the plurality of characteristics corresponding to the respective second drug and the at least one first biological component;
identify a subset comprising at least a first identified node, a second identified node, and a third identified node of the plurality of nodes;
identify a particular characteristic of the subset, the drug of the third node not assigned the particular characteristic; and
store an association between the particular characteristic and at least one of the third identified node and the drug of the third identified node.

16. The system of claim 15, wherein the one or more processors are configured to evaluate the association by:
applying, as input to a molecular docking operation, the drug of the third identified node; and
comparing an output of the molecular docking operation with an expected output corresponding to the particular characteristic.

17. The system of claim 15, wherein the one or more processors are configured to generate the first edge by assigning a weight to the first edge corresponding to a number of same characteristics amongst the at least one first characteristic and the at least one second characteristic with respect to the at least one first target.

18-28. (canceled)

29. A method, comprising:
generating, by one or more processors, a drug-drug similarity network;
determining, by the one or more processors, at least one of a cluster or a community using the drug-drug similarity network; and
determining, by the one or more processors, a repositioning of at least one drug associated with the drug-drug similarity network.

30. The method of claim 29, further comprising determining the repositioning for at least a subset of drugs of the drug-drug similarity network for which a match score between a candidate label of the subset and a label of the at least one of the cluster or the community is less than a threshold match score.

31. The method of claim 29, further comprising performing molecular docking using the determined repositioning.

32. A method, comprising:
generating topological clusters and network communities;
relating each cluster and each community to a pharmacological property or pharmacological action;
identifying, within each topological cluster or modularity class community, a subset of drugs that are not compliant with the cluster or community label;
validating indicated repositionings; and
analyzing molecular docking parameters for previously unaccounted repositionings.
